(19) United States
(12) Patent Application Publication
(10) Pub. No.: US 2022/0185846 A1
(43) Pub. Date: Jun. 16, 2022

(54) METHODS FOR SYNTHESIZING BETA-HOMOAMINO ACIDS

(71) Applicant: Protagonist Therapeutics, Inc., Newark, CA (US)

(72) Inventors: Suresh Kumar MANTHATI, Sunnyvale, CA (US); Ashok BHANDARI, Pleasanton, CA (US); Mohammad Reza MASJEDIZADEH, San Jose, CA (US)

(21) Appl. No.: 17/598,762

(22) PCT Filed: Mar. 27, 2020
(86) PCT No.: PCT/US2020/025468

§ 371 Date: Sep. 27, 2021

Related U.S. Application Data
(60) Provisional application No. 62/825,635, filed on Mar. 28, 2019.

(51) Int. Cl. C07K 14/00; C12P 13/04

(52) U.S. Cl. CPC C07K 14/001; C12P 13/04

(57) ABSTRACT

Methods of making β-homoamino acids as intermediate for synthesis of peptide monmer and dimer α4β7-antagonists are disclosed. The disclosed methods include solid phase and solution phase methods.

### RELATED APPLICATIONS

This application claims priority to U.S. Provisional Patent Appl. No. 62/825,635, filed Mar. 28, 2019, which is incorporated herein by reference in its entirety.

### SEQUENCE LISTING

This application is being filed electronically via EFS-Web and includes an electronically submitted sequence listing in .txt format. The .txt file contains a sequence listing entitled “PRTH_035_02WO_ST25.txt” created on Mar. 26, 2020 and having a size of ˜2 kilobytes. The sequence listing contained in this .txt file is part of the specification and is incorporated herein by reference in its entirety.

### FIELD OF THE INVENTION

The disclosure relates to methods of making b-homoamino acids as intermediates for the synthesis of peptide monomer and dimer α4β7-antagonists.

### BACKGROUND OF THE INVENTION

Integrins are noncovalently associated α/β heterodimeric cell surface receptors involved in numerous cellular processes ranging from cell adhesion and migration to gene regulation (Dubree, et al., Selective α4β7 Integrin Antagonist and Their Potential as Anti-inflammatory Agents, J. Med. Chem. 2002, 45, 3451-3457). Differential expression of integrins can regulate a cell's adhesive properties, allowing different leukocyte populations to be recruited to specific organs in response to different inflammatory signals. If left unchecked, integrins-mediated adhesion process can lead to chronic inflammation and autoimmune disease.

The α4 integrins, α4β1 and α4β7, play essential roles in lymphocyte migration throughout the gastrointestinal tract. They are expressed on most leukocytes, including B and T lymphocytes, where they mediate cell adhesion via binding to their respective primary ligands, vascular cell adhesion molecule (VCAM), and mucosal addressin cell adhesion molecule (MAdCAM), respectively. The proteins differ in binding specificity in that VCAM binds both α4β1 and to a lesser extent α4β7, while MAdCAM is highly specific for α4β7. In addition to pairing with the α4 subunit, the β7 subunit also forms a heterodimeric complex with αE subunit to form αEβ7, which is primarily expressed on intraepithelial lymphocytes (IEL) in the intestine, lung and genitourinary tract. αEβ7 is also expressed on dendritic cells in the gut. The αEβ7 heterodimer binds to E-cadherin on the epithelial cells. The IEL cells are thought to provide a mechanism for immune surveillance within the epithelial compartment. Therefore, blocking αEβ7 and α4β7 together may be a useful method for treating inflammatory conditions of the intestine.

Inhibitors of specific integrin-ligand interactions have been shown effective as anti-inflammatory agents for the treatment of various autoimmune diseases. For example, monoclonal antibodies displaying high binding affinity for α4β7 have displayed therapeutic benefits for gastrointestinal auto-inflammatory/autoimmune diseases, such as Crohn's disease, and ulcerative colitis. Id. However, one of these therapies interfered with α4β1 integrin-ligand interactions thereby resulting in dangerous side effects to the patient. Therapies utilizing a dual-specific small molecule antagonists have shown similar side effects in animal models.

Accordingly, there is a need in the art for integrin antagonist molecules having high affinity for the α4β7 integrin and high selectivity against the α4β1 integrin, as a therapy for various gastrointestinal autoimmune diseases.

Such integrin antagonist molecules and related compositions and methods have been described in WO2014059213. Many of the peptides disclosed in the PCT application include beta-amino acids in their sequence. As a result, there is a need for improved methods of synthesizing such intermediate beta-amino acids. Such improved methods are described herein.

### SUMMARY OF INVENTION

In certain aspects, the invention provides methods of preparing β-amino acids as intermediates for synthesis of pharmacologically active peptides. In one embodiment, the pharmacologically active peptides are α4β7 antagonists, e.g., monomer peptides or dimer peptides comprising two peptides. In a particular embodiment, the β-amino acids are useful to prepare peptides using solution phase peptide synthesis.

In further embodiments of the invention, the peptides are synthesized by solid phase peptide synthesis. In still further embodiments of the invention, the peptides are synthesized by solution phase peptide synthesis.

In certain embodiments, the present invention provides methods of synthesizing β-amino acids according to formula VI:

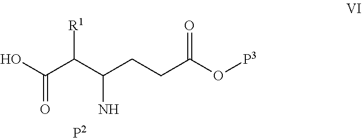

or pharmaceutically acceptable salts, solvates, and hydrates thereof;

wherein

each P¹ and P³ is, independently, an O— protecting group; P² is an N- protecting group; and

R¹ is H, substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol.

In certain embodiments, the method comprises the steps of:

A1) reacting the compound of formula I with 2,2-dimethyl-4,6-dioxo-1,3-dioxane to form

the dioxandione compound of formula II:

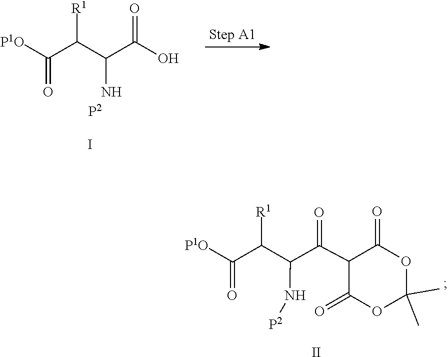

A2) reacting the dioxandione compound of formula II with a reducing agent to obtain the dioxandione compound of formula III:

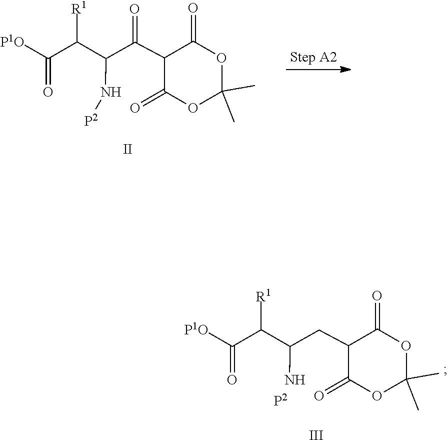

A3) hydrolyzing the dioxandione compound of formula III to form the β-amino acid of formula IV:

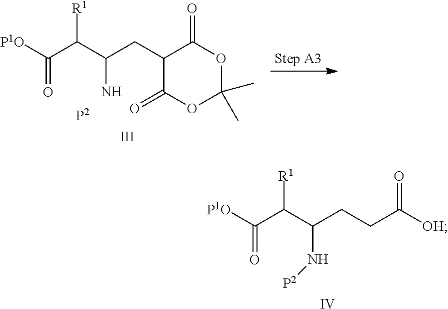

A4) protecting the β-amino acid of formula IV to obtain the protected amino acid of formula V:

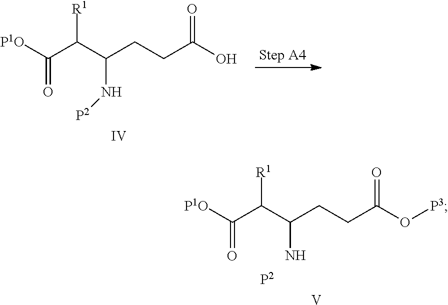

and

A5) reacting the protected amino acid of formula V with a base to form the β-amino acid of formula VI:

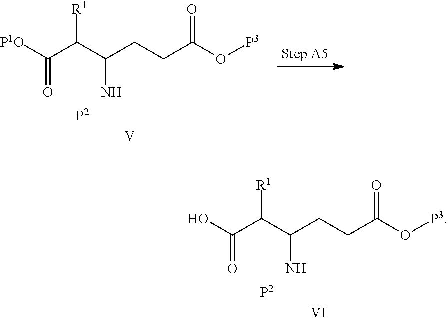

In a particular embodiment, when R¹ is H, P¹ is benzyl, and P³ is t-Bu; then P² is not FMOC.

In a particular aspect, the present invention provides a compound according to formula II:

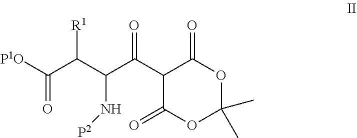

wherein P¹, P², and R¹ as described herein;

provided that when R¹ is H, and P¹ is t-Bu; then P² is other than t-Boc.

In a particular embodiment, when R¹ is H, and P¹ is t-Bu; then P² is not t-Boc.

In another particular aspect, the present invention provides a compound according to formula III:

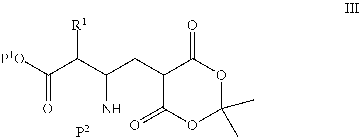

wherein P¹, P², and R¹ as described herein;

provided that when R¹ is H, and P¹ is t-Bu or benzyl; then P² is other than t-Boc.

In a particular embodiment, when R¹ is H, and P¹ is t-Bu; then P² is not t-Boc.

In another particular aspect, the present invention provides a compound according to formula IV:

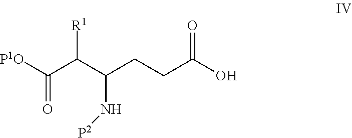

wherein P¹, P², and R¹ as described herein;

provided that

i) when P¹ is Et, and P² is Cbz; then R¹ is H;

ii) when P¹ is benzyl or t-Bu, and P² is t-Boc; then R¹ is other than H;

iii) when P¹ is Me, and P² is benzyl; then R¹ is other than H; and

iv) when P¹ is t-Bu, and P² is FMOC or t-Boc; then R¹ is other than H.

In one embodiment, when P¹ is benzyl or t-Bu, and P² is t-Boc; then R¹ is substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol.

In another embodiment, when P¹ is Me, and P² is benzyl; then R¹ is substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol.

In another embodiment, when P¹ is t-Bu, and P² is FMOC or t-Boc; then R¹ is substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol.

In another particular aspect, the present invention provides a compound according to formula V:

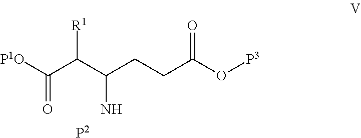

wherein P¹, P², P³, and R¹ as described herein;

provided that when P² is t-Boc, and R¹ is H; then P³ is other than benzyl.

In one embodiment, when P² is t-Boc, and R¹ is H; then P³ is not benzyl.

In another embodiment, when P² is t-Boc, and P³ is benzyl, then R¹ is substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol.

In another particular aspect, the present invention provides a compound according to formula VI:

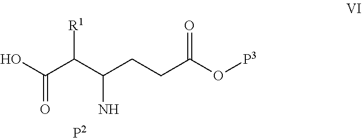

wherein P², P³, and R¹ are as described herein;

provided that when P³ is Me, t-Bu, or benzyl; then R¹ is other than H, OH, or substituted thio.

In one embodiment, when P³ is Me, t-Bu, or benzyl; then R¹ is not H, OH, or substituted thio

In another embodiment, when P³ is Me, t-Bu, or benzyl, then R¹ is substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, or substituted hydroxy.

In one embodiment, with respect to formula II-V, P¹ is benzyl.

In one embodiment, with respect to formula II-VI, P² is Cbz.

In one embodiment, with respect to formula II-VI, R¹ is H.

In one embodiment, with respect to formula II-VI, P³ is t-Bu.

In another aspect, the methods of present invention are used to prepare various homo-amino acids. Such β-amino acids and their precursors are listed in Table 1.

### BRIEF DESCRIPTION OF THE DRAWINGS

FIGS. 1A and 1B depict the MS (M+Na) of Compound of formula VI (P²-Cbz, P³-t-Bu, and R¹-H).

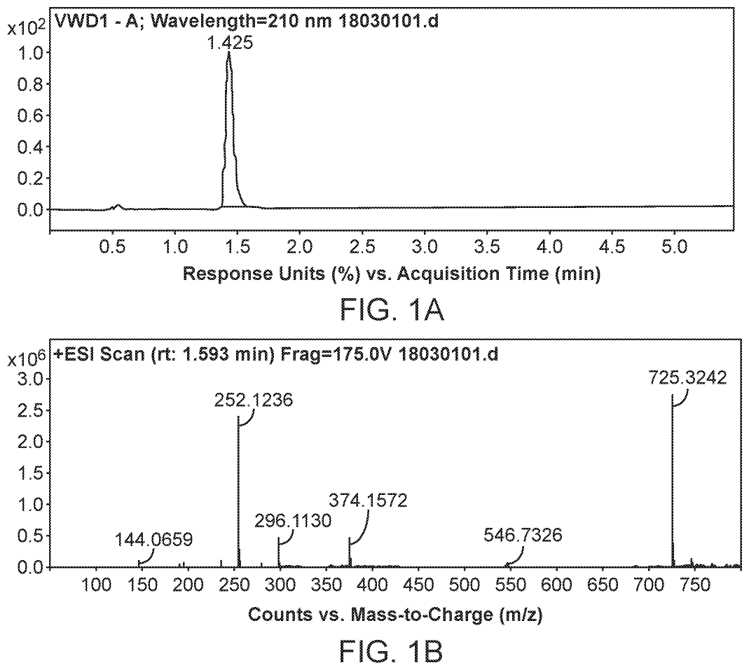

FIG. 2 depicts the ¹H NMR of Compound of formula VI (P²-Cbz, P³-t-Bu, and R¹-H).

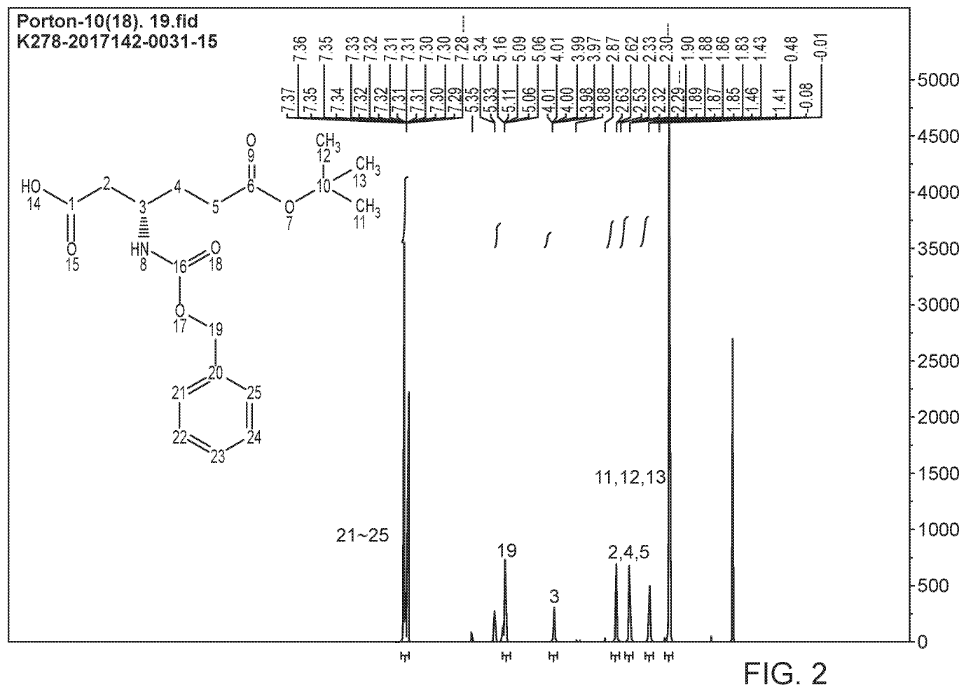

FIG. 3 depicts the ¹³C NMR of Compound of formula VI (P²-Cbz, P³-t-Bu, and R¹— H).

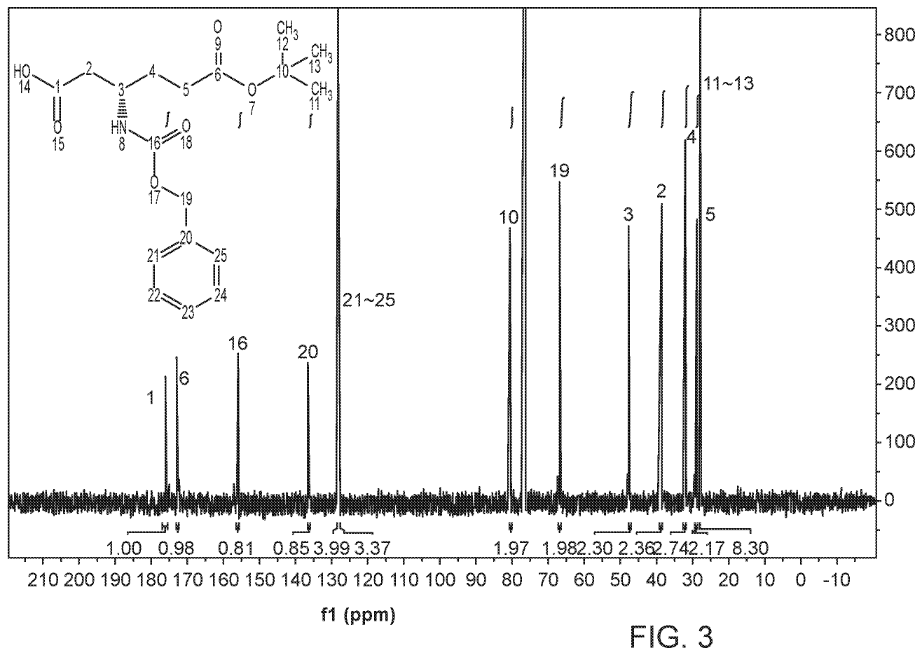

### DETAILED DESCRIPTION OF THE INVENTION

### Definitions

As used herein, the singular forms “a,” “and,” and “the” include plural references unless the context clearly dictates otherwise.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl (n-pr), 1-methylethyl (iso-propyl or i-Pr), n-butyl (n-Bu), n-pentyl, 1,1-dimethylethyl (t-butyl, or t-Bu), 3-methylhexyl, 2-methylhexyl, and the like. Unless stated otherwise specifically in the specification, an alkyl group is optionally substituted by one or more of the following substituents: halo, cyano, nitro, oxo, thioxo, trimethylsilanyl, —OR^{a}, SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

The alkyl group could also be a “lower alkyl” having 1 to 6 carbon atoms.

As used herein, C₁-Cₓ includes C₁-C₂, C₁-C₃ . . . C₁-Cₓ.

“Alkenyl” refers to a straight or branched hydrocarbon chain radical group consisting solely of carbon and hydrogen atoms, containing at least one double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group consisting solely of carbon and hydrogen atoms, containing at least one triple bond, having from two to twelve carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon in the alkylene chain or through any two carbons within the chain. Unless stated otherwise specifically in the specification, an alkylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one double bond and having from two to twelve carbon atoms, for example, ethenylene, propenylene, n-butenylene, and the like. The alkenylene chain is attached to the rest of the molecule through a double bond or a single bond and to the radical group through a double bond or a single bond. The points of attachment of the alkenylene chain to the rest of the molecule and to the radical group can be through one carbon or any two carbons within the chain. Unless stated otherwise specifically in the specification, an alkenylene chain is optionally substituted by one or more of the following substituents: halo, cyano, nitro, aryl, cycloalkyl, heterocyclyl, heteroaryl, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) and —S(O)ₜN(R^{a})₂ (where t is 1 or 2) where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, and where each of the above substituents is unsubstituted unless otherwise indicated.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from six to eighteen carbon atoms, where at least one of the rings in the ring system is fully unsaturated, i.e., it contains a cyclic, delocalized (4n+2) π-electron system in accordance with the Hückel theory. Aryl groups include, but are not limited to, groups such as phenyl (Ph), fluorenyl, and naphthyl. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{β—}OR^{a}, —R^{β—}OC(O)—R^{a}, —R^{β—}N(R^{a})₂, —R^{β—}C(O)R^{a}, —R^{β—}C(O)OR^{a}, —R^{β—}C(O)N(R^{a})₂, —R^{β—}O—R^{c}—C(O)N(R^{a})₂, —R^{β—}N(R^{a})C(O)OR^{a}, —R^{β—}N(R^{a})C(O)R^{a}, —R^{β—}N(R^{a})S (O)ₜR^{a} (where t is 1 or 2), —R^{β—}S(O)ₜOR^{a} (where t is 1 or 2) and —R^{β—}S(O)ₜN(R^{a})₂ (where t is 1 or 2), where each R^{a} is independently hydrogen, alkyl, fluoroalkyl, cycloalkyl, cycloalkylalkyl, aryl (optionally substituted with one or more halo groups), aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, benzyl, diphenylmethyl and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{c}-aryl, where R^{c} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is optionally saturated, (i.e., containing single C-C bonds only) or unsaturated (i.e., containing one or more double bonds or triple bonds.) A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{β}—OR^{a}, —R^{β}—SR^{a}, —R^{β}—OC(O)—R^{a}, —R^{β}—N(R^{a})₂, —R^{β}—C(O)R^{a}, —R^{β}—C(O)OR^{a}, —R^{β}—C(O)N(R^{a})₂, —R^{β}—O—R^{c}—C(O)N(R ^{a})₂, —R^{β}—N(R^{a})C(O)OR^{a}, —R^{β}—N(R^{a})C(O)R^{a}, —R^{β}—N(R^{a})S(O)ₜR^{a} (where t is 1 or 2), —R^{β}—S(O)ₜOR^{a} (where t is 1 or 2) and —R^{β}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocyclyl, heterocyclylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“Halo” or “halogen” refers to bromo, chloro, fluoro or iodo substituents.

The terms “haloalkyl,” “haloalkenyl,” “haloalkynyl” and “haloalkoxy” include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical is optionally substituted as defined above for an alkyl group.

As used herein, the term “non-aromatic heterocycle”, “heterocycloalkyl” or “heteroalicyclic” refers to a non-aromatic ring wherein one or more atoms forming the ring is a heteroatom. A “non-aromatic heterocycle” or “heterocycloalkyl” group refers to a cycloalkyl group that includes at least one heteroatom selected from nitrogen, oxygen and sulfur. The radicals may be fused with an aryl or heteroaryl. Heterocycloalkyl rings can be formed by three to 14 ring atoms, such as three, four, five, six, seven, eight, nine, or more than nine atoms.

Heterocycloalkyl rings can be optionally substituted. In certain embodiments, non-aromatic heterocycles contain one or more carbonyl or thiocarbonyl groups such as, for example, oxo- and thio-containing groups. Examples of heterocycloalkyls include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane. Illustrative examples of heterocycloalkyl groups, also referred to as non-aromatic heterocycles, include:

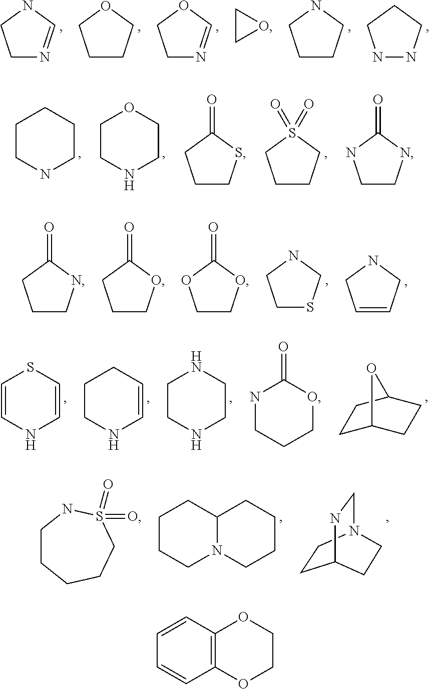

and the like. The term heteroalicyclic also includes all ring forms of the carbohydrates, including but not limited to the monosaccharides, the disaccharides and the oligosaccharides. Depending on the structure, a heterocycloalkyl group can be a monoradical or a diradical (i.e., a heterocycloalkylene group).

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. In some embodiments, heteroaryl rings have five, six, seven, eight, nine, or more than nine ring atoms. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo [1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydro benzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydro cycloocta[d]pyrimidinyl, 5,6,7,8,9,10-hexahydrocycloocta[d]pyridazinyl, 5,6,7,8,9,10-hexa hydrocycloocta[d]pyridinyl,isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetra hydrobenzo[4,5]thieno[2,3-d]pyrimidinyl, 6,7,8,9-tetrahydro-5H-cyclohepta[4,5]thieno [2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno [2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{β}—OR^{a}, —R^{β}—SR^{a}, —R^{β}—OC(O)—R^{a}, —R^{β}—N(R^{a})₂, —R^{β}—C(O)R^{a}, —R^{β}—C(O)OR^{a}, —R^{β}—C(O)N(R^{a})₂, —R^{β}—O—R^{c}—C(O)N(R a)₂, —R^{β}—N(R^{a})C(O)OR^{a}, —R^{β}—N(R^{a})C(O)R^{a}, —R^{β}—N(R^{a})S(O)ₜR^{a} (where t is 1 or 2), —R^{β}—S(O)ₜOR^{a} (where t is 1 or 2) and —R^{β}—S(O)ₜN(R^{a})₂ (where t is 1 or 2), where each R^{a} is independently hydrogen, alkyl, fluoroalkyl, cycloalkyl, cycloalkylalkyl, aryl, aralkyl, heterocycloalkyl, heterocycloalkylalkyl, heteroaryl or heteroarylalkyl, each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Sulfanyl” refers to the —S— radical.

“Sulfinyl” refers to the —S(═O)— radical.

“Sulfonyl” refers to the —S(═O)₂— radical.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Imino” refers to the ═NH radical.

“Thioxo” refers to the ═S radical.

An “alkoxy” group refers to a (alkyl)O— group, where alkyl is as defined herein.

An “aryloxy” group refers to an (aryl)O— group, where aryl is as defined herein.

“Carbocyclylalkyl” means an alkyl radical, as defined herein, substituted with a carbocyclyl group. “Cycloalkylalkyl” means an alkyl radical, as defined herein, substituted with a cycloalkyl group. Non-limiting cycloalkylalkyl groups include cyclopropylmethyl, cyclobutylmethyl, cyclopentylmethyl, cyclohexylmethyl, and the like.

As used herein, the terms “heteroalkyl” “heteroalkenyl” and “heteroalkynyl” include optionally substituted alkyl, alkenyl and alkynyl radicals in which one or more skeletal chain atoms is a heteroatom, e.g., oxygen, nitrogen, sulfur, silicon, phosphorus or combinations thereof. The heteroatom(s) may be placed at any interior position of the heteroalkyl group or at the position at which the heteroalkyl group is attached to the remainder of the molecule. Examples include, but are not limited to, —CH₂—O—CH₃, —CH₂—CH₂—O—CH₃, —CH₂—NH—CH₃, —CH₂—CH₂—NH—CH₃, —CH₂—N(CH₃)—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. In addition, up to two heteroatoms may be consecutive, such as, by way of example, —CH₂—NH—OCH₃ and —CH₂—O—Si(CH₃)₃.

The term “heteroatom” refers to an atom other than carbon or hydrogen. Heteroatoms are typically independently selected from among oxygen, sulfur, nitrogen, silicon and phosphorus, but are not limited to these atoms. In embodiments in which two or more heteroatoms are present, the two or more heteroatoms can all be the same as one another, or some or all of the two or more heteroatoms can each be different from the others.

The term “bond,” “direct bond” or “single bond” refers to a chemical bond between two atoms, or two moieties when the atoms joined by the bond are considered to be part of larger substructure.

An “isocyanato” group refers to a —NCO group.

An “isothiocyanato” group refers to a —NCS group.

The term “moiety” refers to a specific segment or functional group of a molecule. Chemical moieties are often recognized chemical entities embedded in or appended to a molecule.

A “thioalkoxy” or “alkylthio” group refers to a —S-alkyl group.

A “alkylthioalkyl” group refers to an alkyl group substituted with a —S-alkyl group.

As used herein, the term “acyloxy” refers to a group of formula RC(═O)O—.

“Carboxy” means a —C(O)OH radical.

As used herein, the term “acetyl” refers to a group of formula —C(═O)CH₃.

“Acyl” refers to the group —C(O)R.

As used herein, the term “trihalomethanesulfonyl” refers to a group of formula X₃CS(═O)₂— where X is a halogen.

“Cyanoalkyl” means an alkyl radical, as defined herein, substituted with at least one cyano group.

As used herein, the term “N-sulfonamido” or “sulfonylamino” refers to a group of formula RS(═O)₂NH—.

As used herein, the term “O-carbamyl” refers to a group of formula —OC(═O)NR₂.

As used herein, the term “N-carbamyl” refers to a group of formula ROC(═O)NH—.

As used herein, the term “O-thiocarbamyl” refers to a group of formula —OC(═S)NR₂.

As used herein, “N-thiocarbamyl” refers to a group of formula ROC(═S)NH—.

As used herein, the term “C-amido” refers to a group of formula —C(═O)NR₂.

“Aminocarbonyl” refers to a —CONH₂ radical.

As used herein, the term “N-amido” refers to a group of formula RC(═O)NH—.

As used herein, the substituent “R” appearing by itself and without a number designation refers to a substituent selected from among from alkyl, cycloalkyl, aryl, heteroaryl (bonded through a ring carbon) and non-aromatic heterocycle (bonded through a ring carbon).

“Hydroxyalkyl” refers to an alkyl radical, as defined herein, substituted with at least one hydroxy group. Non-limiting examples of a hydroxyalkyl include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl.

“Alkoxyalkyl” refers to an alkyl radical, as defined herein, substituted with an alkoxy group, as defined herein.

An “alkenyloxy” group refers to a (alkenyl)O— group, where alkenyl is as defined herein.

The term “alkylamine” refers to the —N(alkyl)ₓH_{y} group, where x and y are selected from among x=1, y=1 and x=2, y=0. When x=2, the alkyl groups, taken together with the N atom to which they are attached, can optionally form a cyclic ring system.

“Alkylaminoalkyl” refers to an alkyl radical, as defined herein, substituted with an alkylamine, as defined herein.

An “amide” is a chemical moiety with the formula —C(O)NHR or —NHC(O)R, where R is selected from among alkyl, cycloalkyl, aryl, heteroaryl (bonded through a ring carbon) and heteroalicyclic (bonded through a ring carbon). An amide moiety may form a linkage between an amino acid or a peptide molecule and a compound described herein, thereby forming a prodrug. Any amine, or carboxyl side chain on the compounds described herein can be amidified. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

The term “ester” refers to a chemical moiety with formula —COOR, where R is selected from among alkyl, cycloalkyl, aryl, heteroaryl (bonded through a ring carbon) and heteroalicyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “ring system” refers to one, or more than one ring.

The term “membered ring” can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “fused” refers to structures in which two or more rings share one or more bonds.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono- and di-substituted amino groups, and the protected derivatives thereof. By way of example an optional substituents may be L^{S}R^{S}, wherein each L^{S} is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C₁-C₆ alkyl), or -(substituted or unsubstituted C₂-C₆ alkenyl); and each R^{S} is independently selected from H, (substituted or unsubstituted C₁-C₄alkyl), (substituted or unsubstituted C₃-C₆cycloalkyl), aryl, heteroaryl, or heteroalkyl. The protecting groups that may form the protective derivatives of the above substituents are known to those of skill in the art and may be found in references such as Greene and Wuts, above.

The term “peptide,” as used herein, refers broadly to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring.

The term “dimer,” as used herein, refers broadly to a peptide comprising two or more subunits, wherein the subunits are peptides linked at their C- or N-termini. Dimers also include peptides comprising two subunits that are linked via one or more internal amino acid residues or derivatives thereof. Each of the subunits may be linked to the other via its N-terminus, C-terminus, or through an internal amino acid or derivate thereof, which may be different for each of the two subunits. Dimers of the present invention may include homodimers and heterodimers and function as integrin antagonists. Peptide dimer compounds may be described herein using the following nomenclature: [Xₙ]₂, which indicates that the peptide dimer comprises two monomer subunits defined within the brackets (e.g., Xₙ, where X represents an amino acid and n indicates the number of amino acids in the peptide). A linker moiety linking the two peptide subunits may be shown as follows: [Xₙ]₂-λ or λ-[Xₙ]₂, where λ is the linker. Other chemical moieties, such as detectable labels may be shown in a similar manner as for the linker.

The term “L-amino acid,” as used herein, refers to the “L” isomeric form of an amino acid, and conversely the term “D-amino acid” refers to the “D” isomeric form of an amino acid. The amino acid residues described herein are in the “L” isomeric form unless otherwise indicated, however, residues in the “D” isomeric form can be substituted for any L-amino acid residue, as long as the desired function is retained by the peptide.

The term “NH₂,” as used herein, refers to the free amino group present at the amino terminus of a polypeptide or the —CONH₂ group present at the C-terminus of a polypeptide. The term “OH,” as used herein, refers to the free carboxy group present at the carboxy terminus of a peptide. Further, the term “Ac,” as used herein, refers to Acetyl protection through acylation of the N-terminus of a polypeptide, or any amino acid in the peptide. The term “NH₂” may also be used herein to refer to a C-terminal amide group, e.g., in the context of a CONH₂.

The term “carboxy,” as used herein, refers to —CO₂H.

The term “cyclized,” as used herein, refers to a reaction in which one part of a polypeptide molecule becomes linked to another part of the polypeptide molecule to form a closed ring, such as by forming an intramolecular disulfide bridge or other similar bond, e.g. a lactam bond. In particular embodiments, peptide monomer compounds or monomer subunits of peptide dimer compounds described herein are cyclized via an intramolecular bond between two amino acid residues present in the peptide monomer or monomer subunit.

The term “subunit,” as used herein, refers to one of a pair of polypeptide monomers that are joined at the C— or N- terminus to form a dimer peptide composition.

The term “linker,” as used herein, refers broadly to a chemical structure that is capable of linking together a plurality of peptide monomer subunits to form a dimer.

The term “pharmaceutically acceptable salt,” as used herein, represents salts or zwitterionic forms of the compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by treatment of an amino group with a suitable acid. Representative acid addition salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethansulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include, but are not limited to, inorganic acids such as hydrochloric, hydrobromic, sulfuric, and phosphoric, and organic acids such as oxalic, maleic, succinic, and citric. In certain embodiments, any of the peptide momoner compounds or peptide dimer compounds described herein are salt forms, e.g., acetate salts.

The term “N(alpha)Methylation”, as used herein, describes the methylation of the alpha amine of an amino acid, also generally termed as an N-methylation.

All peptide sequences are written according to the generally accepted convention whereby the α-N-terminal amino acid residue is on the left and the α-C-terminal is on the right. As used herein, the term “α-N-terminal” refers to the free α-amino group of an amino acid in a peptide, and the term “α-C-terminal” refers to the free α-carboxylic acid terminus of an amino acid in a peptide. Unless otherwise specified, it is understood that the α-N-terminal residue on the left has a free α-amino group and the α-C-terminal residue on the right has a free α-carboxylic acid group. Peptide sequences may be shown in tables, which may further disclose additional moieties, such as N-terminal or C-terminal chemical modifications, linkers, conjugates, and/or labels, which are present in certain embodiments of the compounds of the invention.

It is noted that the term “comprising” is intended to be open and permits but does not require the inclusion of additional elements or steps. When the term “comprising” is used herein, the term “consisting of” is thus also encompassed and disclosed.

The term “amino acid” or “any amino acid” as used here refers to any and all amino acids, including naturally occurring amino acids (e.g., α -amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The “non-standard,” natural amino acids are pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 amino acids are known to occur naturally and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Some abbreviations useful in describing the invention are defined below in the following Table 1.

The term “isostere” or “isostere replacement,” as used herein, refers to any amino acid or other analog moiety having physiochemical and/or structural properties similar to a specified amino acid. In particular embodiments, an “isostere” or “suitable isostere” of an amino acid is another amino acid of the same class, wherein amino acids belong to the following classes based on the propensity of the side chain to be in contact with polar solvent like water: hydrophobic (low propensity to be in contact with water), polar or charged (energetically favorable contact with water). Illustrative charged amino acid residues include lysine (+), arginine (+), aspartate (−) and glutamate (−). Illustrative polar amino acids include serine, threonine, asparagine, glutamine, histidine and tyrosine. Illustrative hydrophobic amino acids include alanine, valine, leucine, isoleucine, proline, phenylalanine, tryptophane, cysteine and methionine. The amino acid glycine does not have a side chain and is hard to assign to one of the above classes. However, glycine is often found at the surface of proteins, often within loops, providing high flexibility to these regions, and an isostere may have a similar feature. Proline has the opposite effect, providing rigidity to the protein structure by imposing certain torsion angles on the segment of the polypeptide chain. In certain embodiments, an isostere is a derivative of an amino acid, e.g., a derivative having one or more modified side chains as compared to the reference amino acid.

The term “Fmoc peptide synthesis” as used herein refers to the use of Fmoc α-amino (N-terminal) protected amino acids during peptide synthesis. The Fmoc protecting group can be cleaved under mild basic conditions. The side chains of these Fmoc protected amino acids are, as necessary, protected with an appropriate, orthogonal protecting groups that are stable under the mild basic conditions used to cleave the Fmoc protecting group from the N-terminus of the peptide.

The term “Cbz peptide synthesis” refers to the use of Cbz (Z) α-amino (N-terminal) protected amino acids during peptide synthesis. The Cbz protecting group can be cleaved under hydrogenolysis conditions using Pd/C and hydrogen. The side chains of these Cbz protected amino acids are, as necessary, protected with an appropriate, orthogonal protecting groups that are stable under the hydrogenolysis conditions used to cleave the Cbz protecting group from the N-terminus of the peptide.

Compounds described herein include isotopically-labeled compounds, which are identical to those recited in the various formulas and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as ²H, ³H, ¹3C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

TABLE 1
DEFINITIONS AND ABBREVIATIONS

| Abbreviation | Definition |
|---|---|
| 1-1-Indane | 1-Aminoindane-1-carboxylic acid |
| 1-Nal | 1-Napthylalanine |
| 2-2-Indane | 2-Aminoindane-2-carboxylic acid |
| 2-Methly-trifluorobutyric acid | acylated with 2-Methy-4,4,4-Butyric acid |
| 2-Nal | 2-Napthylalanine |
| 3,3-DiphenylAla | 3,3 DiPhenylAlanine |
| 3,3-DiphenylGly | 3,3-DiphenylGlycine |
| Ac- or Ac | Acetyl |
| Acm | Acetamidomethyl |
| Ahx | Aminohexanoic acid |
| Aic | aminoindan-2-carboxylic acid |
| Alloc | Allyloxycarbonyl |
| Aoc | 2-Amino octonoic acid |
| AUC | Area Under Curve |
| Azoc | Azidomethoxycarbonyl |
| Bts | Benzothhiazole-2-sulfonyl |
| Bip | Biphenylalanine |
| Boc | tert-Butyloxycarbonyl |
| Boc-Triazine | Boc-Triazine di-acid |
| Bpoc | (2-(4-Biphenyl)isopropoxycarbonyl) |
| BrPhF | 9-(4-Bromophenyl)-9-fluorenyl |
| Bsmoc | (1,1-Dioxobenzo[b]thiophene-2-yl)methyloxycarbonyl |
| Cav | Cavanine |
| Cba | Cyclobutyl alanine |
| Cbz | Carbobenzyloxy |
| Z | |
| Cit | Citroline |
| CONH₂ | Amide |
| COOH | Acid |
| Cpa | CyclopentylAlanine |
| Cyclobutyl | Cyclobutylalanine |
| Dab | Diaminobutyric acid |
| Dap | Diaminopropionic acid |
| Ddz | 3,5-Dimethoxyphenylisoproxycarbonyl |
| DIC | N,N′-Diisopropylcarbodiimide |
| dNBS | 2,4-Dinitrobenzenesulfonyl |
| Dts | Dithiasuccinoyl |
| DTT | Dithiothreotol |
| Esc | Ethanesulfonylethoxycarbonyl |
| Fmoc | 9-Fluorenylmethyloxycarbonyl |
| Gla | Gama-Carboxy-Glutamic acid |
| Glu(OMe) | L-glutamic acid g-methyl ester |
| HAsp or | homoAspartic acid |
| homoAsp | |
| HBTU | (2-(1H-benzotriazol-1-yl)-1,1,3,3-tetramethyluronium |
| | hexafluorophosphate) |
| HCha | homocyclohexyl Alanine |
| HCys or | homoCysteine |
| homoCys | |
| HFA | Hexafluoroacetone |
| HGlu or | homoGlutamic acid |
| homoGlu | |
| HLys or | homoLysine |
| homoLys | |
| HOBt | 1-hydroxy-benzotriazole |
| HomoLeu or | homoLeucine |
| homoLeu | |
| HPhe | homo Phenylalanine |
| homoPhe | |
| IDA | β-Ala-Iminodiacetic acid (Linker) |
| IDA-Palm | β-Ala (Palmityl)-Iminodiacetic acid |
| ivDde | 1-(4,4-Dimethyl-2,6-dioxocyclohex-1-ylidene)-3- |
| | methylbutyl |
| Me | Methyl |
| NH₂ | Free Amine |
| NHS | N-hydroxysuccinimide |
| pNZ | p-Nitrobenzyloxycarbonyl |
| Nps | 2-Nitrophenylsulfenyl |
| Nsc | 2-(4-Nitrophenylsulfonyl)ethoxycarbonyl |
| Nle | Norleucine |
| N-Me-Arg; | N-Methyl-Arginine |
| N(alpha)Methylation | |
| N-Me-Lys | N-Methyl-Lysine |
| N-Me-Lys (Ac) | N-Methyl-Acetyl-D-lysine |
| MNPPOC | 2-(3,4-methylenedioxy-6-nitrophenyl)proploxycarbonyl |
| O-Me-Tyr | Tyrosine (O-Methyl) |
| oNBS | o-Nitrobenzenesulfonyl |
| oNZ | o-Nitrobenzyloxycarbonyl |
| NVOC | 4-Nitroveratryloxycarbonyl |
| NPPOC | 2-(2-Nitrophenyl)propyloxycarbonyl |
| Orn | Ornithine |
| Pen | Penicillamine |
| Pen(═O) | Penicillamine sulfoxide |
| Phe(2,4-diCl) | (S)-Fmoc-2-amino-3-(2,4-dichlorophenyl)propionic acid |
| Phe(2-carbomyl) | L-2-carbamoylphenylalanine |
| Phe(3,4-diCl) | (S)-Fmoc-2-amino-3-(3,4-dichlorophenyl)propionic acid |
| Phe(3-carbomyl) | L-3-carbamoylphenylalanine |
| Phe(4-carbomyl) | L-4-carbomylphenylalanine |
| Phe(4-CF3) | 4-Trifluoromethyl Phenylalanine |
| Phe(4-COOH) | (4-carboxy-tert-butyl)-L-phenylalanine |
| Phe(4-F) | 4-fluoro-L-phenylalanine |
| Phe(4-Guanidino) or | 4-Guanidine-Phenylalanine |
| 4-Guan | |
| Phe(4-OMe) | (S)-4-methoxyphenylalanine |
| Phe(4-^{t}Bu) | 2-amino-3-(4-tert-butyl-phenyl)propionic acid |
| Pms | 2-[Phenyl(methyl)sulfonio]ethyloxycarbonyl |
| | tetrafluoroborate |
| Poc | Propargyloxycarbonyl |
| Pseudoproline (di methyl) | (^{ψMe,Me}Pro) |
| Pseudoproline | (^{ψH,H}Pro) or (Pro) |
| Sar | Sarcosine |
| Sps | 2-(4-Sulfophenylsulfonyl)ethoxy carbonyl |
| TCP | Tetrachlorophthaloyl |
| TFA | Trifluoroacetic Acid |
| Tic | (3S-)-1,2,3,4-tetrahydroisoquinoline-3-carboxylic acid 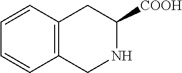 [SMILES: O=C(O)[C@@H]1Cc2ccccc2CN1] |
| TIS | Triisopropylsilane |
| Triazine | Amino propyl Triazine di-acid |
| Trifluorobutyric acid | Acylated with 4,4,4-Trifluorobutyric acid |
| Trifluorpentanoic acid | Acylated with 5,5,5-Trifluoropentanoic acid |
| Troc | 2,2,2-Trichloroethyloxycarbonyl |
| Trt | Triphenylmethyl (Trityl) |
| β-Asp | β-Aspartic acid 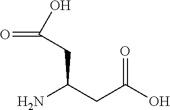 |
| β-HGlu β-homoGlu beta-homoGlu Aad | β-homoglutamic acid 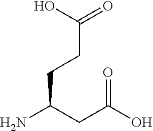 [SMILES: N[C@@H](CCC(=O)O)CC(=O)O] |
| β-HPhe or | β-homophenylalanine |
| β-homoPhe | |
| β-azido-Ala-OH | β-azido-Alanine |
| β-HTrp or | β-homoTrypophane |
| β-homoTrp | |

### β-Amino Acids and Synthesis Thereof

The invention provides methods of preparing key β-amino acids as intermediates for synthesis of pharmacologically active peptides. In one embodiment, the pharmacologically active peptides are α4β7 antagonists. In another embodiment, the β-amino acids are useful to prepare peptides using solution phase peptide synthesis.

In further embodiments of the invention, the peptides are synthesized by solid phase peptide synthesis. In still further embodiments of the invention, the peptides are synthesized by solution phase peptide synthesis.

In certain embodiments, the present invention provides methods of synthesizing β-amino acids according to formula VI:

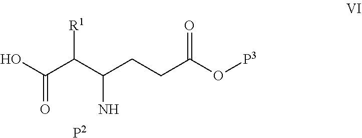

or pharmaceutically acceptable salts, solvates, and hydrates thereof;

wherein

each P¹ and P³ is, independently, an O— protecting group; P² is an N—protecting group; and

R¹ is H, substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol.

In certain embodiments, the method comprises the steps of:
A1) reacting the compound of formula I with 2,2-dimethyl-4,6-dioxo-1,3-dioxane to form
the dioxandione compound of formula II:

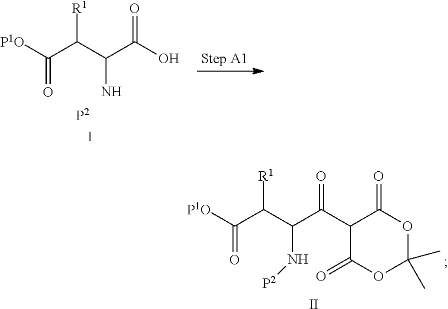

A2) reacting the dioxandione compound of formula II with a reducing agent to obtain the dioxandione compound of formula III:

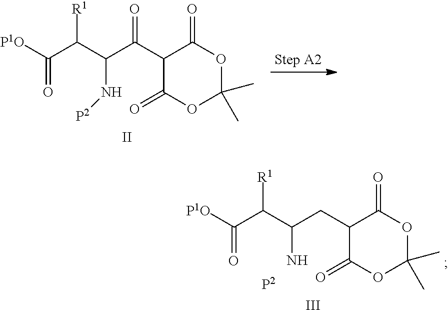

A3) hydrolyzing the dioxandione compound of formula III to form the β-amino acid of formula IV:

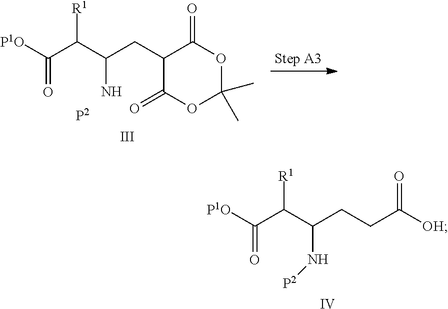

A4) protecting the β-amino acid of formula IV to obtain the protected amino acid of formula V:

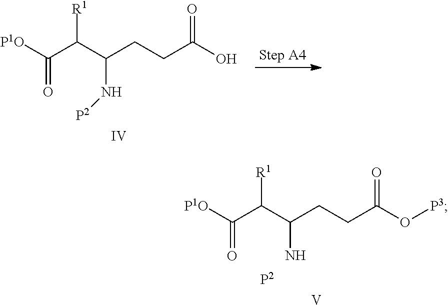

A5) reacting the protected amino acid of formula V with a base to form the β-amino acid of formula VI:

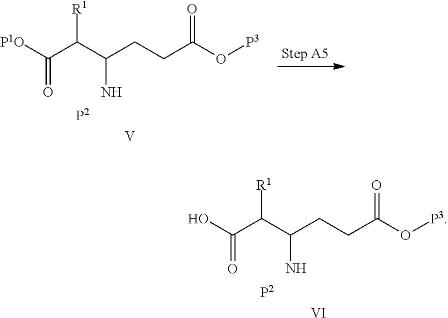

In a particular embodiment, when R¹ is H, P¹ is benzyl, and P³ is t-Bu; then P² is not FMOC.

In particular embodiments, the O-protecting group is any one of the O-protecting groups listed in “Amino Acid—Protecting Groups” by Isidro-Llobet et. al, Chem. Rev. 2009, 109, 2455-2504. Examples of O-protecting groups that may be used include, but are not limited to: Alky esters (the most commonly used are methyl esters, ethyl esters and t-butyl esters) (when P3 is t-butyl, P1 cannot be t-butyl): 9-Fluorenylmethyl esters (9-Fm); 2-(Trimethylsilyl)ethoxymethyl ester (SEM); Methoxyethoxymethyl ester (MEM); Tetrahydropyranyl ester (THP); Benzyloxymethyl ester (BOM); Cyanomethyl ester; Phenacyl ester; 2-(Trimethylsilyl)ethyl ester; Haloester; N-Phthalimidomethyl ester; Benzyl ester; Diphenylmethyl ester; o-Nitrobenzyl ester; Orthoester; and 2,2,2-Trichloroethyl ester.

In particular embodiments, the N-protecting group is any one of the N-protecting groups listed in “Amino Acid—Protecting Groups” by Isidro-Llobet et. al, Chem. Rev. 2009, 109, 2455-2504. Examples of N-protecting groups that may be used include, but are not limited to: 9-Fluorenylmethyl carbamate (Fmoc); 2,2,2-Trichloroethyl carbamate; 2-Trimethylsilylethyl carbamate (Teoc); t-butyl carbamate (Boc) (in some embodiments, when P1 or P3 is t-butyl, P2 cannot be Boc); Allyl carbamate (Alloc); Benzyl carbanate (Cbz); and m-Nitrophenyl carbamate.

In certain embodiments, the step A1 occurs in the presence of a solvent.

In certain embodiments, the step A1 occurs in the presence of methylene chloride, ethylene chloride, tetrachloroethane, 1,2-dichloroethane, N,N-dimethyl formaide, N,N-dimethylacetamide, N-methyl-2-pyrrolidone, dimethylsulfoxide (DMSO), N,N-dimethylformamide (DMF), N,N-dimethylacetamide (DMAc), acetonitrile (MeCN), 1,4-dioxane, tetrahydrofuran (THF), ethyl acetate (EtOAc) or mixtures thereof.

In certain embodiments, the step A1 occurs in the presence of dichloromethane.

In certain embodiments, the step A1 occurs in the presence of a coupling reagent.

In certain embodiments, the step A1 occurs in the presence of diisopropylcarbodiimide (DIC), 1-ethyl-3-(3-dimethylaminopropyl)carbodiimide (EDCI), isopropenyl chloroformate (IPCF), diethyl cyanophosphonate (DEPC), or N,N′-dicyclohexylcarbodiimide (DCC).

In certain embodiments, the step A1 occurs in the presence of 1-ethyl-3-(3-dimethylaminopropyl)carbodiimide (EDCI). In certain embodiments 1-ethyl-3-(3-dimethylaminopropyl)carbodiimide (EDCI) is in a form of hydrochloride (EDCI.HCl).

In certain embodiments, the step A1 occurs in the presence of a base.

In certain embodiments, the step A1 occurs in the presence of DMAP, pyridine or substituted pyridine. In a particular embodiment, the step A1 occurs in the presence of DMAP.

In certain embodiments, the step A1 occurs at 0-50° C.

In certain embodiments, the step A1 occurs at 0-10° C. In certain embodiments, the step A1 occurs at 0-5° C. In a particular embodiment, the step A1 occurs around 0° C.

In certain embodiments, the step A1 occurs for 0.5-18 h.

In certain embodiments, the step A1 occurs for 1-10, 1-5, 1-4, 1-3, 1-2 or about 2 h.

In certain embodiments, the step A1 occurs for about 2 h. In certain embodiments, the step A1 occurs for about 3-10 h. In certain embodiments, the step A1 occurs for about 5-10 h. In certain embodiments, the step A1 occurs for about 7-10 h. In certain embodiments, the step A1 occurs for about 9-10 h. In certain embodiments, the step A1 occurs for about 9 h.

In certain embodiments, the step A2 occurs in the presence of a solvent. In certain embodiments, the step A2 occurs in the absence of a solvent.

In certain embodiments, the step A2 occurs in the presence of methylene chloride, ethylene chloride, tetrachloroethane, 1,2-dichloroethane, acetonitrile (MeCN), 1,4-dioxane, tetrahydrofuran (THF), ethyl acetate (EtOAc), methanol (MeOH), ethanol (EtOH), isopropanol (IPA) or mixtures thereof.

In certain embodiments, the step A2 occurs in the presence of THF. In certain embodiments, the step A2 occurs in the presence of a reducing reagent.

In certain embodiments, the step A2 occurs in the presence of a hydride reagent.

In certain embodiments, the step A2 occurs in the presence of sodium borohydride (NaBH₄), sodium cyanoborohydride (NaCNBH₃), or sodim triacetoxyborohydride (Na(OAc)₃BH).

In a particular embodiment, the step A2 occurs in the presence of sodium borohydride (NaBH₄).

In certain embodiments, the step A2 occurs in the presence of an acid.

In certain embodiments, the step A2 occurs in the presence of a carboxylic acid.

In certain embodiments, the step A2 occurs in the presence of acetic acid, propionic acid, or butyric acid.

In particular embodiments, the step A2 occurs in the presence of acetic acid and sodium borohydride (NaBH₄).

In certain embodiments, the step A2 occurs at 0-100, 0-50, 0-10 or 0-5° C.

In certain embodiments, the step A2 occurs at 0-5° C.

In certain embodiments, the step A2 occurs for 1-24, 2-24, 5-24, 10-24, 15-20, or 16-20 h.

In certain embodiments, the step A2 occurs for 10-15 h. In certain embodiments, the step A2 occurs for 1-5 h.

In certain embodiments, the step A3 occurs in the presence of a solvent.

In certain embodiments, the step A3 occurs in the presence of THF, 2-MeTHF, dioxane, acetonitrile, methyl tert-butyl ether (MTBE), or toluene, or a mixture thereof. In a particular embodiment, the step A3 occurs in the presence of 2-MeTHF.

In certain embodiments, the step A3 occurs in the presence of H₂O.

In certain embodiments, the step A3 occurs at 50-80, 50-75, or 70-75° C.

In certain embodiments, the step A3 occurs at 70-75° C.

In certain embodiments, the step A3 occurs for 1-100, 20-90, 30-70, 40-60, or 50-60 h.

In certain embodiments, the step A3 occurs for 5-20 h. In a particular embodiment, the step A3 occurs for about 12 h.

In certain embodiments, the step A3 occurs for 40-60 h. In certain embodiments, the step A3 occurs for 40-50 h.

In certain embodiments, the step A4 occurs in the presence of a solvent.

In certain embodiments, the step A4 occurs in the presence of methylene chloride, ethylene chloride, tetrachloroethane, dioxane, THF, acetonitrile, methyl tert-butyl ether (MTBE), and toluene. In a particular embodiment, the step A4 occurs in the presence of methylene chloride.

In certain embodiments, the step A4 occurs in the presence of isobutene.

In certain embodiments, the step A4 occurs in the presence of C₁-C₆ alcohol.

In certain embodiments, the step A4 occurs in the presence of MeOH, EtOH, n-PrOH, i-PrOH, or cyclohexanol.

In certain embodiments, the step A4 occurs in the presence of an excess amount of alcohol and in the presence of sulfuric acid, methanesulfonic acid, ethanesulfonic acid, benzenesulfonic acid, p-tolenesulfonic acid or camphorsulfonic acid. In one embodiment, the alcohol used is in excess amount

In certain embodiments, the step A4 occurs in the presence of an excess amount of alcohol and in the presence of methanesulfonic acid.

In certain embodiments, the step A4 occurs in the presence of an stoichometric amount of alcohol and in the presence of a coupling agent. In one embodiment, the coupling agent is any conventional coupling agent used in such reactions. In one embodiment, the coupling agent is EDC, or DCC. In one embodiment, the coupling agent is DIC.

In certain embodiments, the step A4 occurs at −20 to 50, −10 to 20, −10 to 10, or −5 to 10° C.

In certain embodiments, the step A4 occurs at about 0° C.

In certain embodiments, the step A4 occurs for 1-24 h, 1-15, or 5-15 h.

In certain embodiments, the step A4 occurs for 5-15 h. In certain embodiments, the step A4 occurs for about 12 h. In certain embodiments, the step A4 occurs for about 4-5 h.

In particular embodiments, the step A4 occurs in presence of dichloromethane and isobutene, and at −5 to 0° C. for 4-5 h.

In certain embodiments, the step A5 occurs in the presence of a solvent.

In certain embodiments, the step A5 occurs in the presence of methanol, THF, dioxane, 2Me-THF, EtOH, isoPrOH, or water.

In certain embodiments, the step A5 occurs in the presence of methanol. In a particular embodiment, the step A5 occurs in the presence of THF:methanol. In a particular embodiment, the step A5 occurs in the presence of methanol:water.

In certain embodiments, the step A5 occurs in the presence of a base.

In certain embodiments, the step A5 occurs in the presence of aq. NaOH, aq. LiOH, aq. KOH, aq. Ba(OH)2, aq. Na₂CO₃, aq. K₂CO₃, DBU/LiBr, or DBU/LiCl.

In certain embodiments, the step A5 occurs in the presence of aq. LiOH. In certain embodiments, the step A5 occurs in the presence of aq. NaOH. In certain embodiments, the step A5 occurs in the presence of 30% aq. NaOH.

In certain embodiments, the step A5 occurs at 10-50, 15-40, or 20-25° C.

In certain embodiments, the step A5 occurs at 20-25° C.

In certain embodiments, the step A5 occurs for 1-24, 1-10, 2-6, or 4-6 h.

In certain embodiments, the step A5 occurs for 4-6 h. In certain embodiments, the step A5 occurs for 3-4 h.

In certain embodiments, P¹ is benzyl, 4-methoxybenzyl, or 2,4-dimethoxybenzyl.

In certain embodiments, P² is t-Bu. In certain embodiments, P² is methyl, ethyl, iso-propyl, cyclopropyl, or cyclohexyl.

In certain embodiments, P³ is Cbz. In certain embodiments, P³ is Boc, Ddz, Bpoc, Nps, Nsc, Bsmoc, ivDde, TCP, Pms, Esc, Sps, Alloc, oNBS, dNBS, Bts, Troc, Dts, pNZ, Poc, oNZ, NVOC, NPPOC, MNPPOC, BrPhF, Azoc, HFA (Isidro-Llobet, et al., Amino Acid Protecting Groups, Chem. Rev. 2009, 109, 2455-2504).

In certain embodiments, R¹ is substituted or unsubstituted alkyl.

In certain embodiments, R¹ is Me, Et, i-Pr, or t-Bu.

In certain embodiments, R¹ is substituted or unsubstituted aryl.

In certain embodiments, R¹ is substituted or unsubstituted aralkyl.

In certain embodiments, R¹ is substituted or unsubstituted benzyl, naphth-1-ylmethyl, or naphth-2-ylmethyl.

In certain embodiments, R¹ is substituted or unsubstituted benzyl.

In certain embodiments, R¹ is substituted or unsubstituted heteroarylalkyl.

In certain embodiments, R¹ is substituted or unsubstituted imidazomethyl or indolylmethyl.

In certain embodiments, R¹ is substituted or unsubstituted aminoalkyl.

In certain embodiments, R¹ is substituted or unsubstituted aminomethyl, aminoethyl, aminopropyl, or aminobutyl.

In certain embodiments, R¹ is substituted or unsubstituted hydroxymethyl, hydroxyethyl, hydroxypropyl, or hydroxybutyl.

In certain embodiments, R¹ is substituted or unsubstituted thiomethyl, thioethyl, thiopropyl, or thiobutyl.

In certain embodiments, R¹ is substituted or unsubstituted guanidinoalkyl.

In certain embodiments, R¹ is substituted or unsubstituted amino, substituted or unsubstituted hydroxy, or substituted or unsubstituted thiol.

In certain embodiments, R¹ is H.

In a particular aspect, the present invention provides a compound according to formula II:

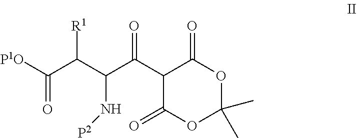

wherein P¹, P², and R¹ as described herein;

provided that when R¹ is H, and P¹ is t-Bu; then P² is other than t-Boc.

In a particular embodiment, when R¹ is H, and P¹ is t-Bu; then P² is not t-Boc.

In one embodiment, with respect to formula II, P¹ is benzyl. In another embodiment, P² is Cbz or C(O)OCH₂Ph. In yet another embodiment, P² is t-Boc, P¹ is t-Bu and R¹ is substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol. In a particular embodiment, P¹ is benzyl, and P² is Cbz. In a more particular embodiment, R¹ is H, P¹ is benzyl, and P² is Cbz.

In another particular aspect, the present invention provides a compound according to formula XII:

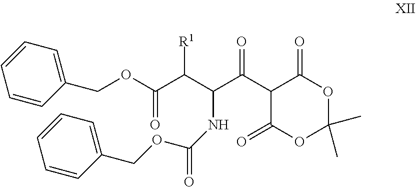

wherein R¹ is as described herein.

In one embodiment, with respect to formula XII, R¹ is H.

In another particular aspect, the present invention provides a compound according to formula III:

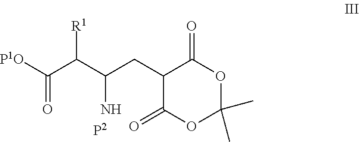

wherein P¹, P², and R¹ as described herein;

provided that when R¹ is H, and P¹ is t-Bu or benzyl; then P² is other than t-Boc.

In one embodiment, when R¹ is H, and P¹ is t-Bu or benzyl; then P² is not t-Boc.

In one embodiment, with respect to formula II, P² is Cbz or C(O)OCH₂Ph. In another embodiment, P² is t-Boc, P¹ is t-Bu or benzyl, and R¹ is substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol. In a particular embodiment, P¹ is benzyl, and P² is Cbz. In a more particular embodiment, R¹ is H, P¹ is benzyl, and P² is Cbz.

In another particular aspect, the present invention provides a compound according to formula XIII:

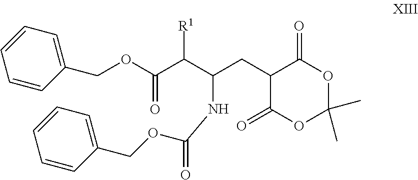

wherein R¹ is as described herein.

In one embodiment, with respect to formula XIII, R¹ is H.

In another particular aspect, the present invention provides a compound according to formula IV:

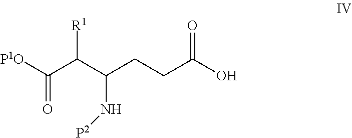

wherein P², and R¹ are as described herein; and P1 is Me, Et, t-Bu, or benzyl; provided that
i) when P¹ is Et, and P² is Cbz; then R¹ is H;
ii) when P¹ is benzyl or t-Bu, and P² is t-Boc; then R¹ is other than H;
iii) when P¹ is Me, and P² is benzyl; then R¹ is other than H; and
iv) when P¹ is t-Bu, and P² is FMOC or t-Boc; then R¹ is other than H.

In one embodiment, P¹ is benzyl, and P² is Cbz. In another embodiment, P¹ is t-Bu, and P² is Cbz. In another embodiment, P¹ is Me, and P² is Cbz.

In another particular aspect, the present invention provides a compound according to formula XIV:

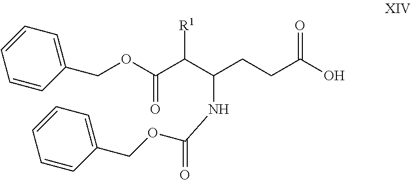

wherein R¹ as described herein.

In one embodiment, with respect to formula XIV, R¹ is H.

In another particular aspect, the present invention provides a compound according to formula V:

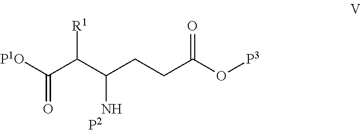

wherein P¹ is benzyl; and P², P³, and R¹ are as described herein;

provided that when P² is t-Boc, and R¹ is H; then P³ is other than benzyl.

In another particular aspect, the present invention provides a compound according to formula XV:

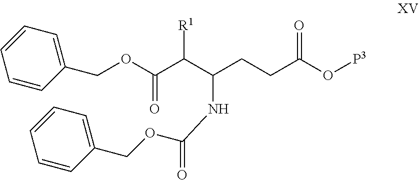

wherein R¹ and P³ are as described herein.

In one embodiment, with respect to formula XV, R¹ is H. In another embodiment, P³ is t-Bu.

In another particular aspect, the present invention provides a compound according to formula VI:

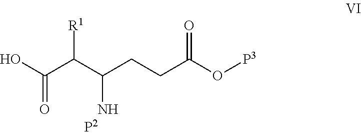

wherein P² is Cbz, and P³, and R¹ as described herein;

provided that when P³ is Me, t-Bu, or benzyl; then R¹ is other than H, OH, or substituted thio.

In one embodiment, with respect to formula II-V, P¹ is benzyl.

In one embodiment, with respect to formula II-VI, P² is Cbz.

In one embodiment, with respect to formula II-VI, R¹ is H.

In one embodiment, with respect to formula II-VI, P³ is t-Bu.

In particular embodiments of the invention, the methods described herein can be used to prepare peptides and peptide dimers on a commercial and/or industrial scale. In particular embodiments of the invention, the methods of the invention can be used to synthesize about 10 to 150 kg of peptide or peptide dimer. In certain embodiments of the invention, the methods described herein can be used to synthesize about 10 to 125 kg, 10 to 100 kg, 10 to 75 kg, 10 to 50 kg, 10 to 25 kg, 25 to 150 kg, 25 to 125 kg, 25 to 100 kg, 25 to 75 kg, 25 to 50 kg, 50 to 150 kg, 50 to 125 kg, 50 to 100 kg, 50 to 75 kg, 75 to 150 kg, 75 to 125 kg, 75 to 100 kg, 100 to 125 kg, 100 to 150 kg, or 125 to 150 kg, 100 to 500 kg, 500-1,000 kg, 1,000 to 10,000 kg, and all subranges there between.

Embodiments of the methods of synthesis disclosed herein can be used to synthesize various β-homoamino acids which in turn can be used to synthesize containing β-homoamino acid peptide monomers and dimers. In particular embodiment, the methods of synthesis disclosed herein can be used to synthesize various β-homoamino acid which are intermediates for β-homoamino acid containing peptide monomers and dimers described in WO2014059213. An illustrative method of synthesizing a peptide is provided in Example 6, which may also be adapted to synthesize other peptides. Certain embodiments of this invention provide feasibility to synthesize on commercial quantities up to multi metric ton scale. Certain embodiments of this invention provide significant advantages; such as simple operations, minimal side reactions, amenable to large scale production. In certain embodiments of the invention, the thiol group of a penicillamine is protected by pseudoproline derivative during solid phase peptide synthesis.

In particular embodiments of the invention, the method provides synthesis of β-homoamino acid which in turn can be used to synthesize the linear decapeptide, Ac-Pen-N(Me)Arg-Ser-Asp-Thr-Leu-Pen-Phe(4-′Bu)-β-homoGlu-D-Lys-NH₂ (SEQ ID NO: 1).

### EXAMPLES

### Example 1

### Synthesis of Amino Acid of Formula VI

### General Peptide Synthesis Protocols

General Procedure for Preparation of N-Cbz Protected Amino Acids:

The amino acid (10.0 g) is dissolved in H₂O (300 ml) and Na₂CO₃ (2.0 equiv) and NaHCO₃ (1.0 equiv) are added at room temperature, with stirring, to give a clear solution. Acetone (4.0 vol, with respect to the amino acid) is added and the slightly turbid solution is cooled in an ice water bath to 15-20° C. Cbz-Cl (1.25 equiv) is added slowly, with stirring, and the reaction mixture allowed to warm to room temperature. After stirring for an additional three hours at room temperature the mixture is extracted with methyl tert-butyl ether (50 ml). To the aqueous phase, 1N aqueous HCl is slowly added to give a pH of 2. The resulting oil is extracted into methyl tert-butyl ether (2×100 mL) and the organic phase is washed with H₂O (100 ml), dried, filtered and concentrated in vacuo to give the N-Cbz protected amino acid as a white solid of viscous-oil.

General Procedure for Condensation:

Cbz-AA-OH (1.2 equiv.), N-hydroxysuccinimide (NHS; 1.2-1.4 equiv.), are suspended in dichloromethane. The resulting slurry is cooled to below 5° C. Then, 1-ethyl-3-(3-dimethylaminopropyl) carbodiimide hydrochloride (EDAC) is added in portions over a period of 30 mins. The resulting clear solution is stirred for 4 hours at 0° C. A solution of HRN-AA-OP (1-1.2 equiv.) in dichloromethane is added over a period of five minutes. The resulting brown solution is stirred at room temperature overnight. The reaction mixture is diluted with water, and the organic phase separated. The organic phase is washed with dilute HCl solution, bicarbonate solution (2 times) and brine. The organic phase is separated, dried, filtered and concentrated to give the peptide.

General Procedure for Deprotection of Cbz:

In an appropriate size round bottom flask or hydrogenation apparatus Cbz-protected compound is dissolved in methanol. The resulting clear solution was purged with argon gas, and catalytic amounts of 10% Pd/C is added. The mixture is stirred under H₂ (1 atm) at RT until no starting material can be detected by TLC analysis. The amine compound is confirmed by developing on TLC and staining with ninhydrin. The catalyst is removed by filtration through a pad of Celite and washed with methanol. The filtrate is concentrated under reduced pressure to give the corresponding amine, which is used in the amide-formation reaction without further purification.

Protected linear decapeptide amide (segment AB, 10) is dissolved in a cold solution of cocktail mixture (0-5° C.) TFA/H2O/TIS (9.0:0.5:0.25) and stirred for two hours. The reaction mass is filtered to remove precipitated product, the solution is concentrated to ¾ volume under reduced pressure and the remaining solution is triturated with isopropyl ether.

### Example 2

### Representative Synthesis of Cbz Protected Amino Acid of Formula F

### Formula VI, p2=Cbz, p3=t-Bu, and R¹ is H

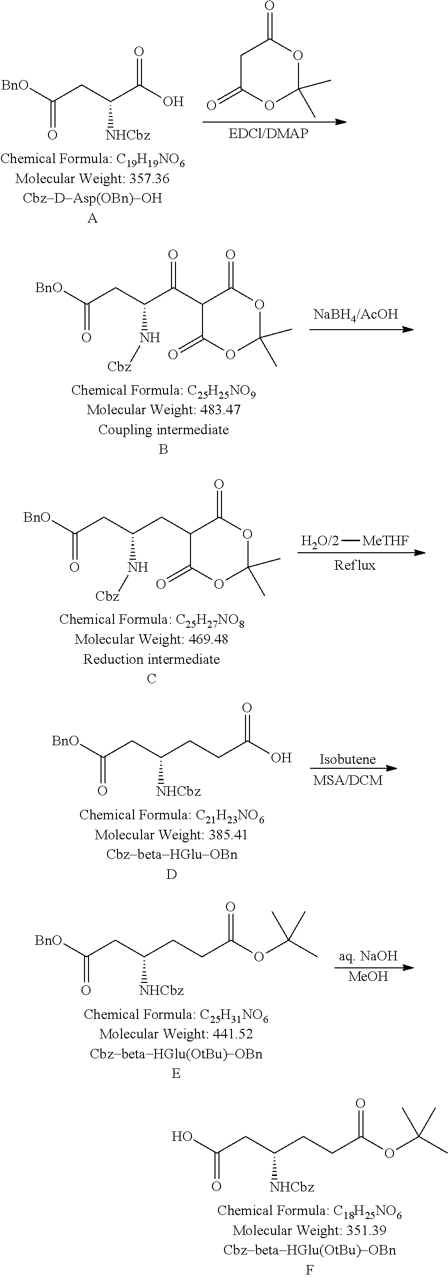

### Step A1: Synthesis of Benzyl(R)-3-(((benzyloxy)carbonyl)amino)-4-(2,2-dimethyl-4,6-dioxo-1,3-dioxan-5-yl)-4-oxobutanoate (B)

A 2-L round-bottomed flask is charged with Cbz-D-Asp(OBn)-OH (285 g, 0.8 mol), Meldrum's acid (144.13 g, 1 mol) and DMAP (12.2 g, 0.1 mol) in DCM (500 mL) at 20˜25° C. The resulting solution is cooled to 0° C., then a solution of EDC (191.0 g, 1 mol) in DCM (500 mL) is added over a period of 10 minutes. The reaction mixture continued to stir for another 2 h. The reaction mixture is diluted with water (500 mL) and dichloromethane (500 mL). The organic phase is separated and washed with 5% phosphoric acid (500 mL), 10% sodium bicarbonate (500 mL) and brine (500 mL). The organic phase is separated, dried, filtered and evaporated to give the title compound as an oil. (362 g, 94% yield).

### Step A2: Synthesis of Benzyl (S)-3-(((benzyloxy)carbonyl)amino-4-(2,2-dimethyl-4,6-dioxo-1,3-dioxan-5-yl)butanoate (C)

A 5-L cylinderical reactor is charged with Cbz-D-Asp(OBn)-OMeldrum's ester (B) (360 g, 0.74 mol) in DCM (1 L). The resulting clear solution is cooled to 0° C., then 122.7 g acetic acid is added. NaBH₄ (37.83 g, 1.0 mol) is added in potions over a period of 1 h. The reaction mixture is stirred for 10-15 h at 0° C. Then the reaction mixture is diluted with 2.9 L 13% aq. KHSO₄. The organic layer is collected and futher washed with 2.7 L H₂O, followed by 2.9 L 13% aq. KHSO₄. The organic phase is dried, filtered and evaporated to give a viscous-oil (328 g, 94% yield).

### Step A3: Synthesis of (S)-6-(Benzyloxy)-4-(((benzyloxy)carbonyl)amino)-6-oxohexanoic acid (D)

A 2-L round-bottom flask is charged with Cbz-D-homo-Asp(OBn)-ester (C) (328 g, 0.7 mol) in 2-MeTHF (500 mL) and water (500 mL). The resulting biphasic reaction mixture is slowly warmed to reflux for 12 h. The organic phase is separated, dried, filtered, and evaporated to give the title compound as off-white solid (250 g, 92.5%).

### Step A4: Synthesis of 1-Benzyl-6-(tert-butyl) (S)-3-(((benzyloxy)carbonyl)amino)-hexanedioate (E)

A 2-L round-bottom flask is charged with Cbz-beta-homoGlu-OBn (D) (250 g, 0.65 mol) in DCM (500 mL). The resulting reaction mixture is cooled to 0° C. and methanesulfonic acid (25 g) and isobutene (561 g) are added. The reaction mixture is stirred for another 12 h at 0° C. The reaction mixture is quenched with water (500 mL). The organic phase is separated, dried, filtered and evaporated to give the title compound as off-white solid (250 g, 87.4%).

### Step A5: Synthesis of (S)-3-(((benzyloxy)carbonyl)amino)-6-(tert-butoxy)-6-oxohexanoic acid (F)

A 2-L round-bottom flask is charged with Cbz-beta-homoGlu(OtBu)-OBn (250 g, 0.56 mol) in methanol (200 mL) and THE (200 mL). The resulting solution is cooled to 0° C. and then sodium hydroxide (15.54 g) in water (50 mL) is added. The reaction mixture is stirred for another 6 h. Then the reaction pH is adjusted to ˜7 and the organic volatiles are removed under vacuum. The reaction pH is slowly adjusted to 2-3 with 6N. HCl. During this period, product precipitated as off-white solid. The product is separated by filtration, dried under vacuum to give the title compound (160 g, 80.4%).

### Example 3

### Representative Synthesis of Cbz Protected Amino Acid of Formula F

### Formula VI, P2=Cbz, P3=t-Bu, and R¹ is H

### Synthesis Using Different P¹ Groups (P¹ is Alkyl, for Example, Me, Et, or Cyclohexyl)

### Step 1. Synthesis of Alkyl (R)-3-((benzyloxy)carbonyl)amino)-4-(2,2-dimethyl-4,6-dioxo-1,3-dioxan-5-yl)-4-oxobutanoate

To a round-bottomed flask charge with Cbz-D-Asp(OP¹)—OH, Meldrum's acid and DMAP in DCM at 20˜25° C. The solution cool to 0° C., then a solution of EDC in DCM is added over a period of 10 minutes. The reaction mixture is continued to stir for another 2 h. The reaction mixture is diluted with water and dichloromethane. The organic phase is separated and washed with 5% phosporic acid, 10% sodium bicarbonate and brine. The organic phase is separated, dried, filtered and evoporated to give the titlecompund as an oil.

### Step 2. Synthesis of Alkyl (S)-3-((benzyloxy)carbonyl)amino-4-(2,2-dimethyl-4,6-dioxo-1,3-dioxan-5-yl)butanoate (C)

In a round-bottom flask charge with Cbz-D-Asp(OP¹)—OMeldrum's ester in DCM. The clear solution is cooled to 0° C., and then acetic acid is added. Add NaBH₄ in potions over a period of 1 h. The reaction mixture is stirred for 10-15 h at 0° C. Then the reaction mixture is diluted with aq. KHSO₄. The organic layer is collected and futher washed with H₂O, followed by aq. KHSO₄, filtered, dried, and evaporated to give viscous-oil.

### Step 3. Synthesis of (S)-6-(Alkyl)-4-((benzyloxy)carbonyl)amino)-6-oxohexanoic acid

A round-bottom flask is charged with Cbz-D-homo-Asp(Oalkyl)-ester in 2-MeTHF and water. The resulting biphasic reaction mixture is slowly warmed to reflux for 12 h. Separate the organic phase, dry, filter and evaporate to give the title compound as off-white solid.

### Step 4. Synthesis of 1-Alkyl-6-(tert-butyl) (S)-3-((benzyloxy)carbonyl)amino)-hexanedioate

A round-bottom flask is charged with Cbz-beta-homoGlu-OAlkyl in DCM. The resulting reaction mixture cools to 0° C. and methane sulfonic acid and isobutene are added. The reaction mixture is stirred for another 12 h at 0° C. Quench the reaction mixture with water. Separate the oranic phase, dry, filter, and evaporate to give the title compound as off-white solid.

### Step 5. Synthesis of (S)-3-((benzyloxy)carbonyl)amino)-6-(tert-butoxy)-6-oxohexanoic acid

To a round-bottom flask charge with Cbz-beta-homoGlu(OtBu)-OAlkyl in methanol and THF. Cool the resulting solution to 0° C. and then add sodium hydroxide in water. Stir the reaction mixture for another 6 h. Then adjust the reaction pH to ˜7 and remove the organic volatiles under vacuum. Adjust the reaction pH to 2-3 with 6N HCl. The product precipitate as off-white solid. The product is separated by filtration, dried under vacuum to give the title compound.

### Example 4

### Representative Synthesis of Cbz Protected Amino Acid of Formula F

### Formula VI, P2=Boc, P3=t-Bu, and R¹ is H

### Synthesis Using Different P¹ Groups (P¹ is Alkyl, for Example, Me, Et, or Cyclohexyl)

### Step 1. Synthesis of Alkyl (R)-3-((tert-butoxycarbonyl)amino)-4-(2,2-dimethyl-4,6-dioxo-1,3-dioxan-5-yl)-4-oxobutanoate

A round-bottomed flask is charged with Boc-D-Asp(OP1)-OH, Meldrum's acid and DMAP in DCM at 20˜25° C. The solution is cooled to 0° C., then a solution of EDC in DCM is added over a period of 10 minutes. The reaction mixture continues to stir for another 2 h. The reaction mixture is diluted with water and dichloromethate. The organic phase is separated and washed with 5% phosporic acid, 10% sodium bicarbonate, and brine. The organic phase is separated dried, filtered and evaporated to give the title compound as an oil.

### Step 2. Synthesis of Alkyl (S)-3-((tert-butoxycarbonyl)amino-4-(2,2-dimethyl-4,6-dioxo-1,3-dioxan-5-yl)butanoate

In a reactor charge with Boc-D-Asp(OP1)-OMeldrum's ester in DCM. The clear solution is cool to 0° C., and then add acetic acid. Add NaBH₄ in potions over a period of 1 h. The reaction mixture is stirred for 10-15 h at 0° C. Then the reaction mixture is diluted with aq. KHSO₄. The organic layer is collected and futher washed with H₂O, followed by aq. KHSO₄, dried, filtered, and evaporated to give viscous-oil.

### Step 3. Synthesis of (S)-6-(Alkyl)-4-((tert-butoxycarbonyl)amino)-6-oxohexanoic acid

A round-bottom flask charge with Boc-D-homo-Asp(Oalkyl)-ester in 2-MeTHF and water. The resulting biphasic reaction mixture slowly warm to reflux for 12 h. Separate the organic phase, dry, filter and evaporate to give the title compound as off-white solid.

### Step 4. Synthesis of 1-Alkyl-6-(tert-butyl) (S)-3-((tert-butoxycarbonyl)amino)-hexanedioate

A round-bottom flask charge with Boc-beta-homoGlu-OAlkyl in DCM. The resulting reaction mixture cool to 0° C. and methanesulfonic acid and isobutene are added. The reaction mixture is stirred for another 12 h at 0° C. Quench the reaction mixture with water. Separate the oranic phase, dry, filter, and evaporate to give the title compound as off-white solid.

### Step 5. Synthesis of (S)-3-((tert-butoxycarbonyl)amino)-6-(tert-butoxy)-6-oxohexanoic acid

To a round-bottom flask charge with Boc-beta-homoGlu(OtBu)-OAlkyl in methanol and THF. Cool the resulting solution and then add sodium hydroxide in water. Stir the reaction mixture for another 6 h. Then the reaction pH adjust to ˜7 and remove the organic volatiles under vacuum. Adjust the reaction pH to 2-3 with 6N. HCl. The product precipitates as off-white solid. The product is separated by filtration, dried under vacuum to give the title compound.

### Example 5

### Representative Synthesis of Cbz Protected Amino Acid of Formula F

### Formula VI, P² is Fmoc, P³ is Me, Et, or cycloheoxyl, and R¹ is H

### Step 1. Synthesis of Benzyloxy (R)-3-(((fluorenylmethyloxy)carbonyl)amino)-4-(2,2-dimethyl-4,6-dioxo-1,3-dioxan-5-yl)-4-oxobutanoate

A round-bottomed flask is charged with Fmoc-D-Asp(OBn)-OH, Meldrum's acid and DMAP in DCM at 20˜25° C. The solution is cooled to 0° C., then a solution of EDC in DCM is added over a period of 10 minutes. The reaction mixture is to stir for another 2 h. The reaction mixture is diluted with water and dichloromethate. The organic phase is separated and washed with 5% phosphoric acid, 10% sodium bicarbonate and brine. The organic phase is separated, dried, filtered and evaporated to give the title compound as an oil.

### Step 2. Synthesis of Benzoyloxy (S)-3-(((fluorenylmethyloxy)carbonyl)amino-4-(2,2-dimethyl-4,6-dioxo-1,3-dioxan-5-yl)butanoate

A round-bottom flask is charged with Fmoc-D-Asp(OBn)-OMeldrum's ester in DCM. The clear solution is cooled to 0° C., and then add acetic acid. Add NaBH₄ is added in portions over a period of 1 h. The reaction mixture is stirred for 10-15 h at 0° C. Then the reaction mixture is diluted with aq. KHSO₄. The organic layer is collected and futher washed with H₂O, followed by aq. KHSO₄, dried, filtered, and evaporated to give viscous-oil.

### Step 3. Synthesis of (S)-6-(Benzyl)-4-(((fluorenylmethyloxy)carbonyl)amino)-6-oxohexanoic acid

A round-bottom flask is charged with Fmoc-D-homo-Asp(OBenzyl)-ester in 2-MeTHF and water. The resulting biphasic reaction mixture is slowly warmed to reflux for 12 h. The organic phase is separated, dried, filtered and evaporated to give the title compound as off-white solid.

### Step 4. Synthesis of 1-Benzyl-6-(alkyl) (S)-3-(((fluorenylmethyloxy)carbonyl)amino)-hexanedioate

A round-bottom flask is charged with Fmoc-β-homoGlu-OBenzyl in DCM. The resulting reaction mixture is cooled to 0° C. and methane sulfonic acid and alcohol (methyl, ethyl or cyclohexyl) are added. The reaction mixture is stirred for another 12 h at RT. The reaction mixture is quenched with water. The organic phase is separated, drired, filtered, and evaporated to give the title compound as off-white solid.

### Step 5. Synthesis of (S)-3-(((fluorenylmethyloxy)carbonyl)amino)-6-(alkyl)-6-oxohexanoic acid

A round-bottom flask is charged with Fmoc-β-homoGlu(Oalkyl)-OBn in methanol and THF. The solution is subjected to hydrogenation in presence of Pd catlyst. The catalyst is separated by filtration to give the title compound.

### Example 6

### Solid Phase Synthesis of Compound A with Pen (Acm)

A peptide dimer compound, Compound A, comprising two peptide monomers linked at their respective C-termini by a diglycolic acid (DIG) linker was synthesized as described below.

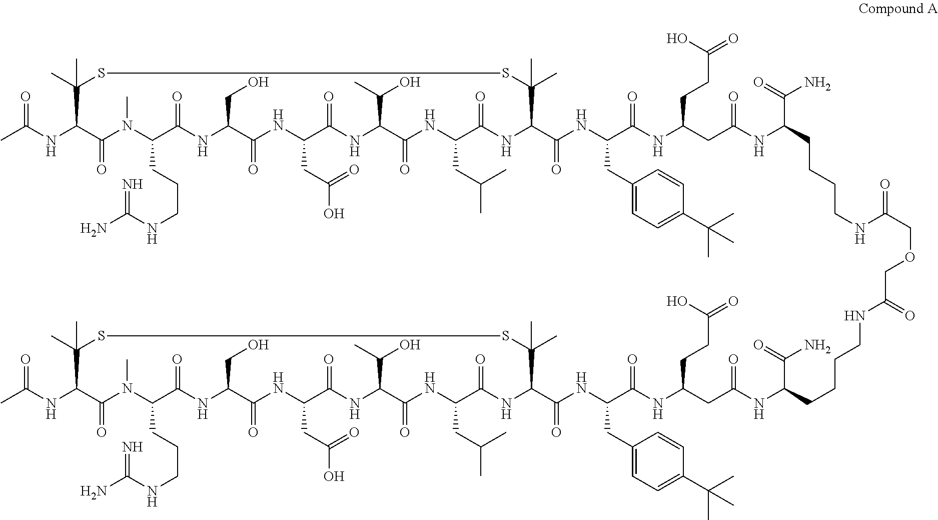 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCNC(=O)COCC(=O)NCCCC[C@@H](NC(=O)C[C@H](CCC(=O)O)NC(=O)[C@H](Cc2ccc(C(C)(C)C)cc2)NC(=O)[C@H]2NC(=O)[C@H](CC(C)C)NC(=O)[C@H](C(C)O)NC(=O)[C@H](CC(=O)O)NC(=O)[C@H](CO)NC(=O)[C@H](CCCNC(=N)N)N(C)C(=O)[C@@H](NC(C)=O)C(C)(C)SSC2(C)C)C(N)=O)C(N)=O)C(C)(C)SSC1(C)C]

Peptide Sequence Assembly

The monomer peptide sequence Ac-Pen-N(Me)Arg-Ser-Asp-Thr-Leu-Pen-Phe(4-′Bu)-β-homoGlu-(D)Lys-NH₂ (SEQ ID NO:1) was assembled by standard solid phase peptide synthesis techniques as follows with the starting materials described in Table 2.

Solid phase synthesis was performed on a tricyclic amide linker resin (DL-form, 200-400 mesh, 0.6 mmol/g loading, 18.0 mmol scale). Approximately 2 equivalents of the Fmoc-proteted amino acid was combined with 3.0 eq Oxyma (Ethyl (hydroxyimino)cyanoacetate) and 2.6 eq DIC (N,N′-Diisopropylcarbodiimide in DMF), and after 20 minutes of stirring the activated amino acid was added to the resin. After 20 minutes an extra 1.4 eq of DIC was added to the coupling solution in the reactor and the coupling reaction proceeded for approximately 1.3 hour to 2.0 hours. The coupling reaction was monitored by removing a sample of the resin from the reactor, washing it multiple times in a micro filtration syringe with DMF and IPA, and performing an appropriate clorimetric test for the specific amino acid. Fmoc-deprotection was performed using a solution of 20/80 piperidine/DMF.

Pen(Acm) was coupled as follows: 2.0 eq amino acid, 2.2 eq oxyma, and 2.0 eq DIC in 50:50 DCM:DMF were allowed to react for 20 minutes, after which the activated amino acid was transferred to the reactor and allowed to react for approximately 48 hrs at room temperature. The reaction was monitored by the Chloranil test.

Pen(Trt) was coupled as follows: 2.0 eq amino acid, 2.2 eq oxyma, and 2.0 eq DIC in 50:50 DCM:DMF were allowed to react for 20 minutes, after which the activated amino acid was transferred to the reactor and allowed to react for approximately 72 hrs at room temperature. The reaction was monitored by the Chloranil test.

After the final Pen(Acm) was coupled (coupling #10), Fmoc-deprotection was performed and the N-terminus of Pen(Acm) was capped with acetic anhydride. The resulting fully protected resin was washed with DMF and Isopropanol (IPA) and dried under vacuum.

After the final Pen(Trt) was coupled (coupling #10), Fmoc-deprotection was performed and the N-terminus of Pen(Trt) was capped with acetic anhydride. The resulting fully protected resin was washed with DMF and Isopropanol (IPA) and dried under vacuum.

TABLE 2
Starting Materials for Peptide Synthesis

| | | Process |
|---|---|---|
| Starting Material | Structure | Step |
| Tricyclic Amide linker resin (Ramage Resin) | 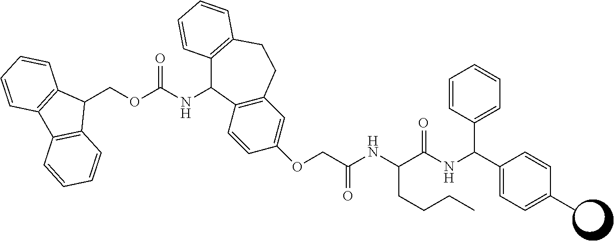 [SMILES: CCCCC(NC(=O)COc1ccc2c(c1)CCc1ccccc1C2NC(=O)OCC1c2ccccc2-c2ccccc21)C(=O)NC(c1ccccc1)c1ccc(C)cc1] | base resin |
| Fmoc-D-Lys(Boc)-OH | 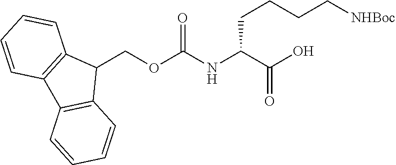 [SMILES: CC(C)(C)OC(=O)NCCCC[C@@H](NC(=O)OCC1c2ccccc2-c2ccccc21)C(=O)O] | coupling #1 |
| Fmoc-L-Aad(O^{t}Bu)-OH (also known as Fmoc-β- HomoGlu(O^{t}Bu)-OH) | 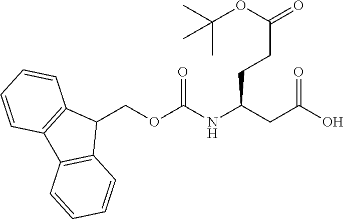 [SMILES: CC(C)(C)OC(=O)CC[C@@H](CC(=O)O)NC(=O)OCC1c2ccccc2-c2ccccc21] | coupling #2 |
| Fmoc-L-(4-^{t}Bu)Phe-OH | 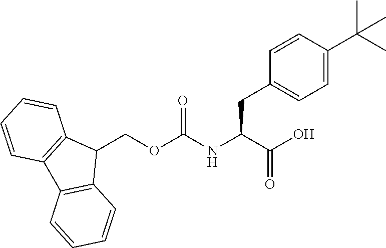 [SMILES: CC(C)(C)c1ccc(C[C@H](NC(=O)OCC2c3ccccc3-c3ccccc32)C(=O)O)cc1] | coupling #3 |
| Fmoc-L-Pen(Acm)-OH | 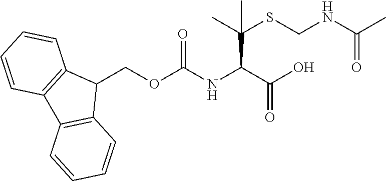 [SMILES: CC(=O)NCSC(C)(C)[C@H](NC(=O)OCC1c2ccccc2-c2ccccc21)C(=O)O] | couplings # 4 and # 10 |
| Fmoc-L-Pen(Trt)-OH | 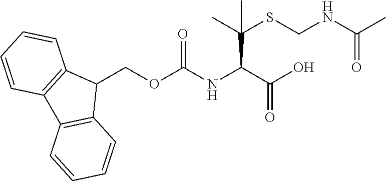 [SMILES: CC(=O)NCSC(C)(C)[C@H](NC(=O)OCC1c2ccccc2-c2ccccc21)C(=O)O] | couplings # 4 and # 10 |
| Fmoc-L-Leu-OH | 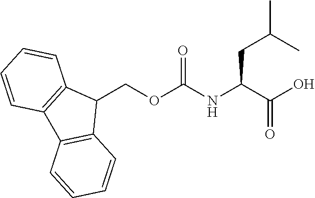 [SMILES: CC(C)C[C@H](NC(=O)OCC1c2ccccc2-c2ccccc21)C(=O)O] | coupling #5 |
| Fmoc-L-Thr(^{t}Bu)OH | 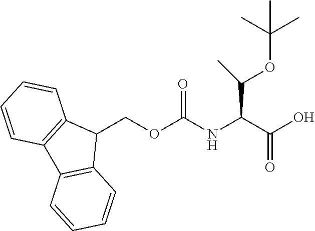 [SMILES: CC(OC(C)(C)C)[C@H](NC(=O)OCC1c2ccccc2-c2ccccc21)C(=O)O] | coupling #6 |
| Fmoc-L-Asp(^{t}Bu)-OH | 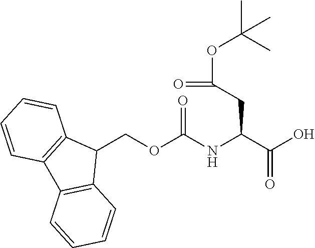 [SMILES: CC(C)(C)OC(=O)C[C@H](NC(=O)OCC1c2ccccc2-c2ccccc21)C(=O)O] | coupling #7 |
| Fmoc-L-Ser(^{t}Bu)-OH | 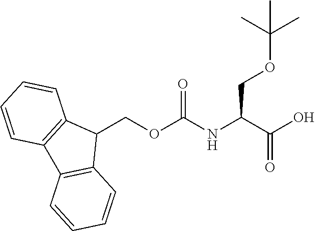 [SMILES: CC(C)(C)OC[C@H](NC(=O)OCC1c2ccccc2-c2ccccc21)C(=O)O] | coupling #8 |
| Fmoc-L-NMe-Arg(Pbf)-OH | 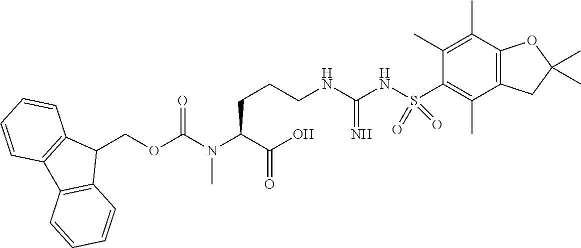 [SMILES: Cc1c(C)c(S(=O)(=O)NC(=N)NCCC[C@@H](C(=O)O)N(C)C(=O)OCC2c3ccccc3-c3ccccc32)c(C)c2c1OC(C)(C)C2] | coupling #9 |
| Acetic anhydride (Ac₂O) | 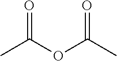 | final capping |

Cleavage and Isolation of Monomer

To cleave the monomer peptide from the resin and to remove side chain protecting groups on the peptide, the protected peptide resin was treated with a cleavage solution containing TFA:water:EDT:TIPS (87.5v:3.5v:8v:1v). The cleavage solution was chilled in the ice bath and thawed to room temperature before use. The cleavage reaction mixture was stirred for about 2 hrs at room temperature. The spent resin was filtered off and washed with a 90:10 mixture of TFA:water. The combined filtrates and washes were then precipitated into cold ethyl ether and centrifuged to collect the peptide. The ethyl ether was decanted, and the solid precipitate was washed three times with cold ethyl ether. The unpurified linear monomer was dried to constant weight under vacuum. TFA cleavage of this peptide resin resulted in a peptide with an Acm-protected Pen residues.

The unpurified monomer was analyzed by RP-HPLC Method 20-40-20 min (Phenomenex Aeris PEPTIDE 3.6 μ XB—C18 150×4.6 mm column), MPA: 0.1% TFA in water and MPB: 0.1% TFA in ACN). LC/MS was performed to verify the expected molecular weight of the linear monomer, and the observed MW of the main product was 1524.5±2 Da.

Disulfide Bond Formation Pen(Acm)

The unpurified linear monomer was dissolved (3.0 gram scale) in 50:50 ACN:water, then diluted to 20:80 ACN:water at a concentration of 2 to 3 mg/mL. While stirring with a magnetic stirrer, a I₂/MeOH solution was added until the solution turned dark yellow. When the yellow color faded out, additional I₂/MeOH solution was added until the reaction mixture stayed a dark yellow to amber color. The reaction was monitored using LCMS and HPLC. When the reaction is completed (uncyclized monomer≤5% (Area %), approximately 30 to 45 minutes), the reaction was quenched with ascorbic acid until a colorless solution was obtained. The reaction mixture was diluted with water (final solution ˜10:90 ACN:water) and purified as discussed below.

Disulfide Bond Formation Pen(Trt)

The unpurified linear monomer was dissolved (3.0 gram scale) in 50:50 ACN:water, then diluted to 20:80 ACN:water at a concentration of 2 to 3 mg/mL. While stirring with a magnetic stirrer, a I₂/MeOH solution was added until the solution turned light yellow. When the yellow color faded out, additional I₂/MeOH solution was added until the reaction mixture stayed a yellow to amber color. The reaction was monitored using LCMS and HPLC. When the reaction is completed (uncyclized monomer≤5% (Area %), approximately 30 to 45 minutes), the reaction was quenched with ascorbic acid until a colorless solution was obtained. The reaction mixture was diluted with water (final solution ˜10:90 ACN:water) and purified as discussed below.

The unpurified cyclized monomer was analyzed by RP-HPLC Method 20-40-20 min (Phenomenex Luna 3.0 μ XB—C18 150×4.6 mm column), MPA: 0.1% TFA in water and MPB: 0.1% TFA in ACN). LC/MS was performed to verify the expected molecular weight of the linear monomer, and the observed MW of the main product was 1381.2±2 Da.

Purification of Cyclized Monomer (Compound B)

The cyclized monomer (Compound B) was purified on a preparative RP-HPLC system using the following conditions: Buffer A: 0.1% TFA in water and Buffer B: 0.1% TFA in ACN, Phenomenex Luna 10 μ C18 250×50 mm column with a flow rate of 80 mL/min. Approximately 3.0 g cyclized monomer was purified per run using a 23:35:60 min gradient (23% B to 35% B in 60 min). Fractions were collected (about 25 fractions per purification, ˜40 mL per fraction) and analyzed by analytical HPLC Method 20-40-20 min and lyophilized. Fractions of purity ≥90% combined for dimerization, fraction with purity between 65 and 90 Area-% were combined for recycling, and fractions with purity<65 Area-% were discarded.

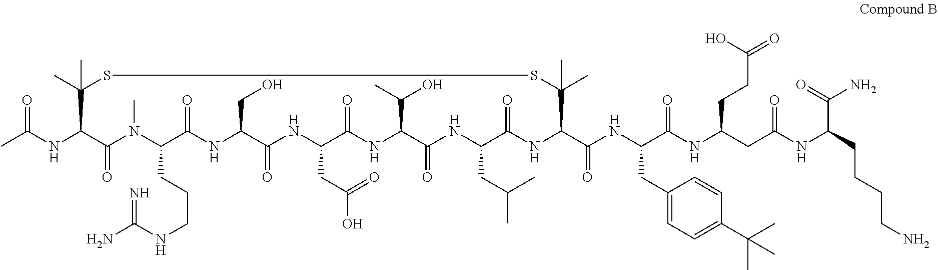 [SMILES: CC(=O)N[C@@H]1C(=O)N(C)[C@@H](CCCNC(=N)N)C(=O)N[C@@H](CO)C(=O)N[C@@H](CC(=O)O)C(=O)N[C@@H](C(C)O)C(=O)N[C@@H](CC(C)C)C(=O)N[C@H](C(=O)N[C@@H](Cc2ccc(C(C)(C)C)cc2)C(=O)N[C@@H](CCC(=O)O)CC(=O)N[C@H](CCCCN)C(N)=O)C(C)(C)SSC1(C)C]

The purified monomer was analyzed by RP-HPLC Method 20-40-20 min (Phenomenex Luna 3.0 μ XB—C18 150×4.6 mm column), MPA: 0.1% TFA in water and MPB: 0.1% TFA in ACN). LC/MS was performed to verify the expected molecular weight of the linear monomer, and the observed MW of the main product was 1381.8±2 Da.

Linker Activation

Diglycolic acid-di-N-Hydroxysuccinimide ester (DIG-OSu₂) was prepared by reacting DIG (Diglycolic acid) (1.0 eq) with HO-Su (N-Hydroxysuccinimide) (2.2 eq) and DCC (N,N′-Dicyclohexylcarbodiimide) (2.2 eq) in NMP for 12 hours at a concentration of 0.1 M. After 12 hrs reaction, the precipitated dicyclohexylurea was removed by filtration, and the DIG-OSu₂ solution (0.1 M) was used for dimerization.

Monomer Dimerization

The cyclized pure monomer was converted to the corresponding dimer by coupling ˜2 g monomer with 0.1 M DIG linker solution (0.45 eq) and DIEA in DMF solution (5.0 eq). The dimerization reaction took approximately 15 to 30 min under ambient conditions. The reaction was monitored using LCMS and HPLC. When the reaction is completed (monomer≤5% (Area %)), the reaction was quenched by adding acetic acid, diluted it with water and purified as discussed below.

The crude dimer (Compound A) was analyzed by the analytical HPLC Method 2-50-20 min (Phenomenex Luna 5 μ C18 150×4.6 mm, 5 micron 100A column), MPA: 0.1% TFA in water and MPB: 0.1% TFA in ACN). LC/MS was used to verify the expected molecular weight of the dimer, and the observed MW was 2859.3±2 Da.

Purification of Compound a and Preparation of the Acetate Salt of Compound A.

The crude dimer was purified on a preparative RP-HPLC system using the following conditions: Buffer A: 0.1% TFA in water and Buffer B: 0.1% TFA in ACN, Phenomenex Luna 10 μ C18 250×50 mm column with a flow rate of 80 mL/min. Approximately 2.0 g dimer was purified per run using a 33:40:60 min gradient (33% B to 40% B in 60 min). Fractions were collected (about 15 fractions per purification, ˜20 mL per fraction) and analyzed by analytical HPLC Method 2-50-20 min. Fraction with purity≥95.0 Area-% were combined as a final product and transferred to salt exchange step (Section 1.6), fractions between 70 and 94 Area-% were combined for recycling, and fractions with purity<60 Area-% were discarded.

The combined purified solution of Compound A from above was diluted with water (1:1) and loaded to a preparative RP-HPLC system using the following conditions: Buffer A: 0.2% AcOH in water and Buffer B: 0.2% AcOH in ACN, Phenomenex Luna 10 μ C18 250×50 mm column with a flow rate of 80 mL/min. Approximately 2.0 g of dimer was loaded per run, after loading the salt exchange step was performed by passing through the column a solution of 0.1 M ammonium acetate, and the material eluted with 0.2% AcOH in ACN. The exchanged fractions were collected and analyzed by analytical HPLC Method 2-50-20 min. Fraction with purity≥95.0 Area-% were combined as a final product, fractions with purity<95 Area-% were re-purified. Fractions were lyophilized using acetate only lyophilizer.

The final purified dimer was analyzed by RP-HPLC Method 22-42-50 min (Phenomenex Aeris PEPTIDE 3.6 μ XB—C18 150×4.6 mm column), MPA: 0.1% TFA in water and MPB: 0.1% TFA in ACN). LC/MS was performed to verify the expected molecular weight of the purified dimer, and the observed MW of the main product was 2859.3±2 Da.

All publications, patents, and patent applications described herein are hereby incorporated by reference in their entireties.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed is:

1. A process for preparing a β-amino acid according to formula VI: 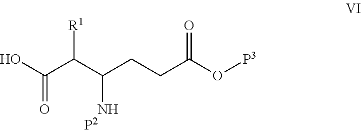
or a pharmaceutically acceptable salt, stereoisomer, isotopic variant or tautomer thereof;
wherein
each P¹ and P³ is independently an O— protecting group; P² is an N—protecting group; and
R¹ is H, substituted or unsubstituted alkyl, substituted or unsubstituted aryl, substituted or unsubstituted cycloalkyl, substituted or unsubstituted aralkyl, substituted or unsubstituted heteroarylalkyl, substituted or unsubstituted aminoalkyl, substituted or unsubstituted hydroxyalkyl, substituted or unsubstituted thiolalkyl, substituted or unsubstituted guanidinoalkyl, substituted or unsubstituted amino, substituted or unsubstituted hydroxy, or substituted or unsubstituted thiol;
wherein the process comprises the steps of
A1) reacting the compound of formula I with 2,2-dimethyl-4,6-dioxo-1,3-dioxane to form
the dioxandione compound of formula II: 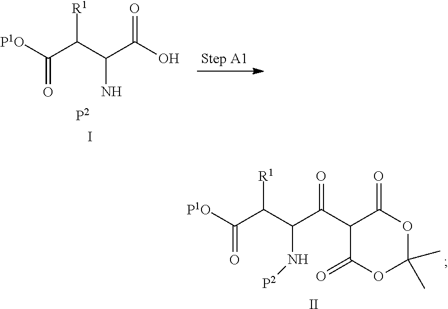
A2) reacting the dioxandione compound of formula II with a reducing agent to obtain the dioxandione compound of formula III: 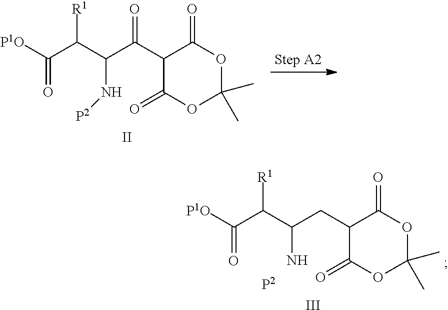
A3) hydrolyzing the dioxandione compound of formula III to form the β-amino acid of formula IV: 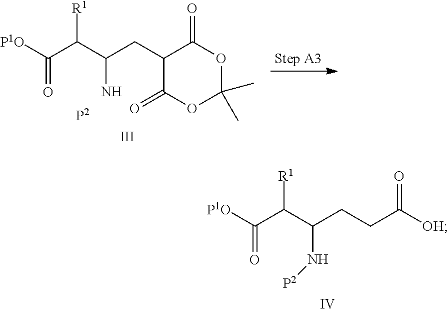
A4) protecting the β-amino acid of formula IV to obtain the protected amino acid of formula V: 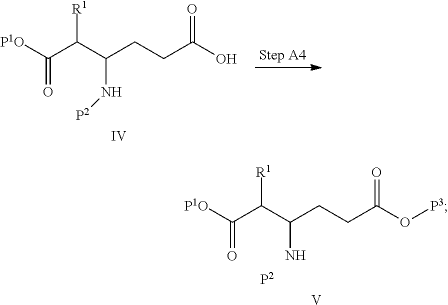
and
A5) reacting the protected amino acid of formula V with a base to form the β-amino acid of formula VI: 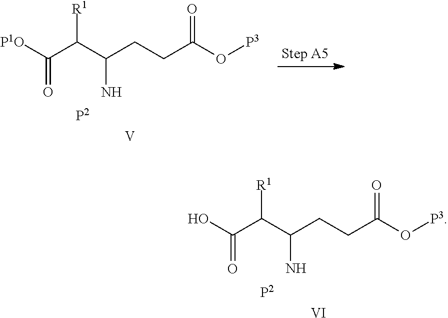
provided that when R¹ is H, P¹ is benzyl, and P³ is t-Bu; then P² is not FMOC.

2. The process according to claim 1, wherein the step A1 occurs in the presence of a solvent.

3. The process according to claim 2, wherein the step A1 occurs in the presence of methylene chloride (DCM), ethylene chloride, tetrachloroethane, 1,2-dichloroethane, N,N-dimethyl formaide, N,N-dimethylacetamide, N-methyl-2-pyrrolidone, dimethylsulfoxide (DMSO), N,N-dimethylformamide (DMF), N,N-dimethylacetamide (DMAc), acetonitrile (MeCN), 1,4-dioxane, tetrahydrofuran (THF), ethyl acetate (EtOAc) or mixtures thereof.

4. The process according to claim 3, wherein the step A1 occurs in the presence of DCM.

5. The process according to any one of claims 1-4, wherein the step A1 occurs in the presence of a coupling reagent.

6. The process according to any one of claims 1-4, wherein the step A1 occurs in the presence of diisopropylcarbodiimide (DIC), 1-ethyl-3-(3-dimethylaminopropyl)carbodiimide (EDCI), isopropenyl chloroformate (IPCF), or diethyl cyanophosphonate (DEPC).

7. The process according to any one of claims 1-4, wherein the step A1 occurs in the presence of 1-ethyl-3-(3-dimethylaminopropyl)carbodiimide (EDCI) or EDCI.HCl.

8. The process according to any one of claims 1-7, wherein the step A1 occurs in the presence of a base.

9. The process according to any one of claims 1-7, wherein the step A1 occurs in the presence of DMAP, pyridine or substituted pyridine.

10. The process according to any one of claims 1-9, wherein the step A1 occurs at 0-50° C.

11. The process according to claim 10, wherein the step A1 occurs at 0-10° C.

12. The process according to any one of claims 1-11, wherein the step A1 occurs for 0.5-18 h.

13. The process according to claim 12, wherein the step A1 occurs for 8-10 h.

14. The process according to any one of claims 1-4, wherein the step A1 occurs in the presence of 1-ethyl-3-(3-dimethylaminopropyl)carbodiimide (EDCI) hydrochloride or EDCI.HCl, and at 0-5° C. for about 9 h.

15. The process according to any one of claims 1-14, wherein the step A2 occurs in the presence of a solvent.

16. The process according to any one of claims 1-14, wherein the step A2 occurs in the presence of methylene chloride, ethylene chloride, tetrachloroethane, 1,2-dichloroethane, acetonitrile (MeCN), 1,4-dioxane, tetrahydrofuran (THF), ethyl acetate (EtOAc), methanol (MeOH), ethanol (EtOH), isopropanol (IPA) or mixtures thereof.

17. The process according to any one of claims 1-14, wherein the step A2 occurs in the presence of THF.

18. The process according to any one of claims 1-14, wherein the step A2 occurs in the presence of a reducing reagent.

19. The process according to any one of claims 1-14, wherein the step A2 occurs in the presence of a hydride reagent.

20. The process according to any one of claims 1-14, sodium borohydride (NaBH4), sodium cyanoborohydride (NaCNBH3), or sodim triacetoxyborohydride (Na(OAc)3BH).

21. The process according to any one of claims 1-14, wherein the step A2 occurs in the presence of an acid.

22. The process according to any one of claims 1-14, wherein the step A2 occurs in the presence of a carboxylic acid.

23. The process according to any one of claims 1-14, wherein the step A2 occurs in the presence of acetic acid, propionic acid, or butyric acid.

24. The process according to any one of claims 1-14, wherein the step A2 occurs at 0-100, 0-50, 0-10 or 0-5° C.

25. The process according to any one of claims 1-14, wherein the step A2 occurs at 0-5° C.

26. The process according to any one of claims 1-14, wherein the step A2 occurs for 5-24, 10-24, 15-20, or 16-20 h.

27. The process according to any one of claims 1-14, wherein the step A2 occurs in the presence of acetic acid and sodium borohydride (NaBH₄), and at 0-5° C. for 1-5 h.

28. The process according to any one of claims 1-27, wherein the step A3 occurs in the presence of a solvent.

29. The process according to any one of claims 1-27, wherein the step A3 occurs in the presence of THF, 2-MeTHF, dioxane, acetonitrile, methyl tert-butyl ether (MTBE), or toluene, or a mixture thereof.

30. The process according to any one of claims 1-27, wherein the step A3 occurs in the presence of H₂O.

31. The process according to any one of claims 1-27, wherein the step A3 occurs at 50-80, 50-75, or 70-75° C.

32. The process according to any one of claims 1-27, wherein the step A3 occurs at 70-75° C.

33. The process according to any one of claims 1-27, wherein the step A3 occurs for 1-100, 20-90, 30-70, 40-60, or 50-60 h.

34. The process according to any one of claims 1-27, wherein the step A3 occurs in the presence of 2-MeTHF, and at 70-75° C. for 40-50 h.

35. The process according to any one of claims 1-34, wherein the step A4 occurs in the presence of a solvent.

36. The process according to any one of claims 1-34, wherein the step A4 occurs in the presence of methylene chloride, ethylene chloride, tetrachloroethane, dioxane, THF, acetonitrile, methyl tert-butyl ether (MTBE), and toluene.

37. The process according to any one of claims 1-34, wherein the step A4 occurs in the presence of isobutene.

38. The process according to any one of claims 1-34, wherein the step A4 occurs in the presence of sulfuric acid, methanesulfonic acid, ethanesulfonic acid, benzenesulfonic acid, p-tolenesulfonic acid or camphorsulfonic acid.

39. The process according to any one of claims 1-34, wherein the step A4 occurs in the presence of methanesulfonic acid.

40. The process according to any one of claims 1-34, wherein the step A4 occurs at −20 to 50° C., −10 to 20° C., −10 to 10° C., or −5 to 10° C.

41. The process according to any one of claims 1-34, wherein the step A4 occurs at about 0° C.

42. The process according to any one of claims 1-34, wherein the step A4 occurs for 1-24 h, 1-15, or 5-15 h.

43. The process according to any one of claims 1-34, wherein the step A4 occurs in presence of dichloromethane and isobutene, and at −5 to 0° C. for 4-5 h.

44. The process according to any one of claims 1-43, wherein the step A5 occurs in the presence of a solvent.

45. The process according to any one of claims 1-43, wherein the step A5 occurs in the presence of methanol, THF, dioxane, 2Me-THF, EtOH, isoPrOH, or water.

46. The process according to any one of claims 1-43, wherein the step A5 occurs in the presence of methanol or methanol:water.

47. The process according to any one of claims 1-43, wherein the step A5 occurs in the presence of a base.

48. The process according to any one of claims 1-43, wherein the step A5 occurs in the presence of aq. NaOH, aq. LiOH, aq. Ba(OH)2, aq. K2CO3, DBU/LiBr, or DBU/LiCl.

49. The process according to any one of claims 1-43, wherein the step A5 occurs in the presence of aq. LiOH.

50. The process according to any one of claims 1-43, wherein the step A5 occurs at 10-50° C., 15-40° C., or 20-25° C.

51. The process according to any one of claims 1-43, wherein the step A5 occurs at 20-25° C.

52. The process according to any one of claims 1-43, wherein the step A5 occurs for 1-24, 1-10, 2-6, or 4-6 h.

53. The process according to any one of claims 1-43, wherein the step A5 occurs in the presence of methanol:water and aq. NaOH, and at 20-25° C. for 3-4 h.

54. The process according to any one of claims 1-53, wherein P¹ is benzyl.

55. The process according to any one of claims 1-54, wherein P² is t-Bu.

56. The process according to any one of claims 1-55, wherein P³ is Cbz.

57. The process according to any one of claims 1-56, wherein R¹ is substituted or unsubstituted alkyl.

58. The process according to any one of claims 1-57, wherein R¹ is Me, Et, i-Pr, or t-Bu.

59. The process according to any one of claims 1-57, wherein R¹ is substituted or unsubstituted aryl.

60. The process according to any one of claims 1-57, wherein R¹ is substituted or unsubstituted aralkyl.

61. The process according to any one of claims 1-57, wherein R¹ is substituted or unsubstituted benzyl, naphth-1-ylmethyl, or naphth-2-ylmethyl.

62. The process according to any one of claims 1-57, wherein R¹ is substituted or unsubstituted benzyl.

63. The process according to any one of claims 1-57, wherein R¹ is substituted or unsubstituted heteroarylalkyl.

64. The process according to any one of claims 1-57, wherein R¹ is substituted or unsubstituted imidazomethyl or indolylmethyl.

65. The process according to any one of claims 1-57, wherein R¹ is substituted or unsubstituted aminoalkyl.

66. The process according to any one of claims 1-57, wherein R¹ is substituted or unsubstituted aminomethyl, aminoethyl, aminopropyl, or aminobutyl.

67. The process according to any one of claims 1-57, wherein R¹ is substituted or unsubstituted hydroxymethyl, hydroxyethyl, hydroxypropyl, or hydroxybutyl.

68. The process according to any one of claims 1-57, wherein R¹ is substituted or unsubstituted thiomethyl, thioethyl, thiopropyl, or thiobutyl.

69. The process according to any one of claims 1-57, wherein R¹ is substituted or unsubstituted guanidinoalkyl.

70. The process according to any one of claims 1-57, wherein R¹ is substituted or unsubstituted amino, substituted or unsubstituted hydroxy, or substituted or unsubstituted thiol.

71. The process according to any one of claims 1-57, wherein R¹ is H.

72. A compound according to formula II: 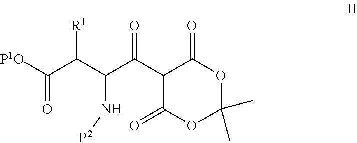
wherein P¹, P², and R¹ are as defined in claim 1;
provided that when R¹ is H, and P¹ is t-Bu; then P² is other than t-Boc.

73. The compound according to claim 72, wherein P¹ is benzyl.

74. The compound according to claim 72, wherein P² is Cbz.

75. The compound according to claim 72, wherein R¹ is H.

76. A compound according to formula XII: 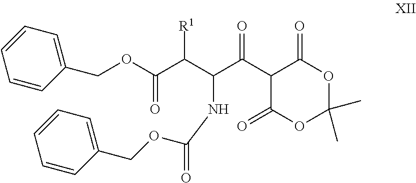
wherein R¹ is as defined in claim 1;

77. The compound according to claim 76, wherein R¹ is H.

78. A compound according to formula III: 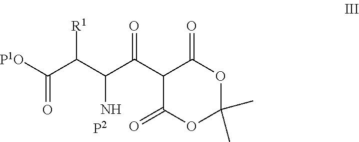
wherein P¹, P², and R¹ are as defined in claim 1;
provided that when R¹ is H, and P¹ is t-Bu or benzyl; then P² is other than t-Boc.

79. The compound according to claim 78, wherein P¹ is benzyl.

80. The compound according to claim 78, wherein P² is Cbz.

81. The compound according to claim 78, wherein R¹ is H.

82. A compound according to formula XIII: 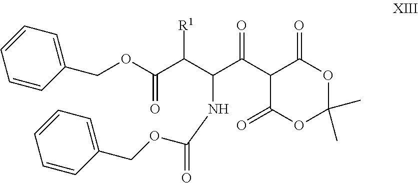
wherein R¹ is as defined in claim 1;

83. The compound according to claim 82, wherein R¹ is H.

84. A compound according to formula IV: 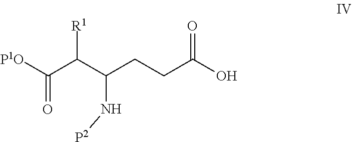
wherein P¹ is Me, Et, t-Bu, or benzyl; and P², and R¹ are as defined in claim 1;
provided that
i) when P¹ is Et, P² is Cbz; then R¹ is H;
ii) when P¹ is benzyl or t-Bu, and P² is t-Boc; then R¹ is other than H;
iii) when P¹ is Me, and P² is benzyl; then R¹ is other than H; and
iv) when P¹ is t-Bu, and P² is FMOC or t-Boc; then R¹ is other than H.

85. The compound according to claim 84, wherein P¹ is benzyl.

86. The compound according to claim 84, wherein P² is Cbz.

87. The compound according to claim 84, wherein P² is Cbz; and P1 is benzyl, t-Bu, or Me.

88. The compound according to claim 84, wherein R¹ is H.

89. A compound according to formula XIV: 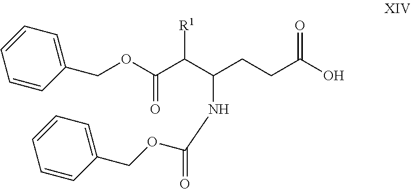
wherein R¹ is as defined in claim 1;

90. The compound according to claim 89, wherein R¹ is H.

91. A compound according to formula V: 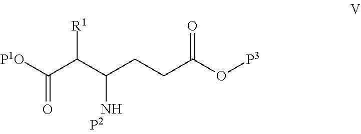
wherein P¹ is benzyl; and P², P³, and R¹ are as defined in claim 1;
provided that when P² is t-Boc, and R¹ is H; then P³ is other than benzyl.

92. The compound according to claim 91, wherein P² is Cbz.

93. The compound according to claim 91, wherein R¹ is H.

94. The compound according to claim 91, wherein P³ is t-Bu.

95. A compound according to formula XV: 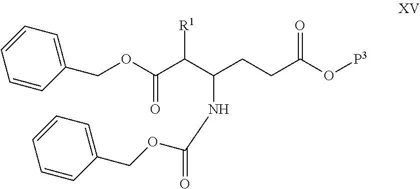
wherein R¹ and P³ are as defined in claim 1.

96. The compound according to claim 95, wherein R¹ is H.

97. The compound according to claim 95, wherein P³ is t-Bu.

98. A compound according to formula VI: 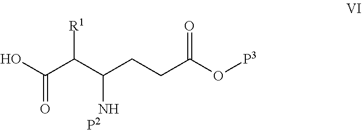
wherein P² is Cbz, and P³, and R¹ are as defined in claim 1;
provided that when P³ is Me, t-Bu, or benzyl; then R¹ is other than H, OH, or substituted thio.

99. The use of the process according to any one of claims 1-71 or the compound according to any one of claims 72-98 in preparation of a peptide.

100. The process according to any one of claims 1-71, wherein the O-protecting group is selected from the group consisting of: Alky esters (optionally, methyl esters, ethyl esters and t-butyl esters); 9-Fluorenylmethyl esters (9-Fm); 2-(Trimethylsilyl)ethoxymethyl ester (SEM); Methoxyethoxymethyl ester (MEM); Tetrahydropyranyl ester (THP); Benzyloxymethyl ester (BOM); Cyanomethyl ester; Phenacyl ester; 2-(Trimethylsilyl)ethyl ester; Haloester; N-Phthalimidomethyl ester; Benzyl ester; Diphenylmethyl ester; o-Nitrobenzyl ester; Orthoester; and 2,2,2-Trichloroethyl ester.

101. The process according to any one of claims 1-71, wherein the N-protecting group is selected from the group consisting of: 9-Fluorenylmethyl carbamate (Fmoc); 2,2,2-Trichloroethyl carbamate; 2-Trimethylsilylethyl carbamate (Teoc); t-butyl carbamate (Boc) (in some embodiments, when P1 or P3 is t-butyl, P2 cannot be Boc); Allyl carbamate (Alloc); Benzyl carbanate (Cbz); and m-Nitrophenyl carbamate.


SEQUENCE LISTING (1 sequence)

SEQ ID NO: 1 - protein, 10 aa, Artificial Sequene
  Made in Lab - Synthesized peptide
  MOD_RES (1)..(1): Penicillamine (Pen)
  MOD_RES (2)..(2): N-methyl-Arginine
  MOD_RES (7)..(7): Penicillamine (Pen)
  MOD_RES (8)..(8): 2-amino-3-(4-tert-butyl-phenyl)propionic acid (Phe(4-tBu))
  MOD_RES (9)..(9): Beta-homoglutamic acid (beta-homoGlu)
  MOD_RES (10)..(10): D form lysine
XXSDTLXXXK